(19) United States
(12) Patent Application Publication
(10) Pub. No.: US 2021/0324037 A1
(43) Pub. Date: Oct. 21, 2021

(54) CHIMERIC MOLECULES

(71) Applicant: ETH Zürich, Zürich (CH)

(72) Inventors: Jan KISIELOW, Schlieren (CH); Franz Josef OBERMAIR, Oberengstringen (CH); Manfred KOPF, Zürich (CH)

(21) Appl. No.: 17/285,338

(22) PCT Filed: Oct. 18, 2019
(86) PCT No.: PCT/EP2019/078449

§ 371 Date: Apr. 14, 2021

(30) Foreign Application Priority Data
Oct. 19, 2018 (EP) 18201560.2

(51) Int. Cl. C07K 14/73; C07K 14/705; G01N 33/68

(52) U.S. Cl. CPC C07K 14/70514; C07K 14/70517; A61K 38/00; G01N 33/68; C07K 2319/00; C07K 14/70596

(57) ABSTRACT

The present invention relates to a method for simultaneous detection and enrichment of antigen-specific T cells and of the peptides specifically recognized by their T cell receptors (TCRs). The method also allows identification of T cell-specific antigens for in vivo and/or in vitro interventions including vaccination, induction of immunological tolerance, blocking of TCRs and MHC-mediated toxin delivery, for immunogenicity testing and other in vitro T-cell reactivity tests. The present invention also relates to the chimeric molecules used in said methods.

The present invention relates to a method for simultaneous detection and enrichment of antigen-specific T cells and of the peptides specifically recognized by their T cell receptors (TCRs). The method also allows identification of T cell-specific antigens for in vivo and/or in vitro interventions including vaccination, induction of immunological tolerance, blocking of TCRs and MHC-mediated toxin delivery, for immunogenicity testing and other in vitro T-cell reactivity tests. The present invention also relates to the chimeric molecules used in said methods.

As medicine in affluent societies enters a new era of personalisation, most therapies and medical products are being progressively tailored for the individual patient. In the case of immunotherapies, antigenic specificities of T-cells come into focus and their unbiased, efficient identification becomes of central importance.

In particular, the development of tumor-specific antigen (TSA)-based cancer vaccines is among the major goals of modern medicine.

Tumor-specific peptides, so-called neoantigens, are only present in tumor cells and entirely absent from the normal tissue. Such tumor-specific peptides are created by tumor-specific DNA alterations that result in the formation of novel protein sequences. Tumor-specific peptides are displayed in the context of the MHC on the surface of tumor cells and so-called antigen presenting cells (APCs). APCs such as dendritic cells or macrophages, can internalize antigens by phagocytosis or by receptor-mediated endocytosis. MHC molecules play an important role in presenting cellular antigens in the form of short linear peptides to T cells. They interact with T cell receptors (TCRs) present on the surface of T cells, which leads to T cell activation. The MHC consists of alpha and beta chains, and a peptide bound in a groove formed by these chains. The stability of this complex is highly dependent on peptide binding, so that empty MHC molecules are downregulated from the cell surface and degraded. Nevertheless, empty MHC molecules exist on the surface of cells and can bind extracellular peptides and present them to T cells.

Once tumor-specific peptides are presented on the cell surface, the cell can be recognized and destroyed by the immune system. Thus, vaccines containing tumor-specific peptides can stimulate the immune system to detect and destroy cancer cells that present these molecules on their surface.

T-cell receptors (TCRs) bind peptide-major histocompatibility complexes (pMHC) with low affinity, posing a considerable challenge for direct identification of T-cell cognate peptides (epitopes). Several different approaches have been developed to solve this problem, but all suffer from a major shortcoming: they have severely limited parallel processing and do not allow simultaneous screening of many TCRs against many epitopes. The TCRs of interest have to be screened against epitope libraries one by one. Vice-versa, finding TCRs reactive to epitopes of interest, requires the epitopes to be screened against TCR libraries one by one.

There is therefore a need for providing means and methods for simultaneous detection and enrichment of antigen-specific T cells and the peptides specifically recognized by their TCRs, and for the identification of T cell-specific antigens for in vivo and/or in vitro interventions. The present invention now satisfies this need in that it provides such means and methods, which are more specifically defined in the claims and the following embodiments of the invention.

### EMBODIMENTS OF THE INVENTION

A1. A chimeric molecule comprising the CD4, LAGS or CD8 co-receptor protein and a peptide attached to the N-terminus of the co-receptor.
A2. The chimeric molecule of embodiment A1, wherein the peptide or a part of the peptide can be presented by a major histocompatibility complex.
A3. The chimeric molecule of embodiments A1 or A2, wherein the peptide has a length of 6 to 200 amino acid residues.
A4. The chimeric molecule of any one of embodiments A1 to A3, wherein the peptide has a length of 7 to 30 amino acids residues.
A5. The chimeric molecule of any one of embodiments A1 to A4, wherein the peptide is a random peptide.
A6. The chimeric molecule of any one of embodiments A1 to A5, wherein the peptide is a peptide that is encoded by a given DNA or cDNA molecule.
A7. The chimeric molecule of embodiment A6, wherein the DNA or cDNA molecule encoding the peptide is obtained by fragmentation of a larger DNA or cDNA molecule.
A8. The chimeric molecule of any one of embodiments A1 to A7, wherein the peptide is derived from a tumor cell or from a cell that has been infected with a pathogen.
A9. The chimeric molecule of any one of embodiments A1 to A8, wherein the peptide comprises an epitope of a tumor antigen.
A10. The chimeric molecule of embodiment A9, wherein the to or antigen is a neoantigen.
A11. The chimeric molecule of any one of embodiments A1 to A10, wherein the peptide comprises an amino acid sequence with at least 50%, 60%, 70%, 80%, 90% sequence identity with an epitope of a tumor antigen.
A12. The chimeric molecule of any one of embodiments A1 to A11, wherein the peptide comprises an MHC class I epitope when the co-receptor protein is CD8.
A13. The chimeric molecule of any one of embodiments A1 to A12, wherein the peptide comprises an MHC class II epitope when the co-receptor protein is CD4 or LAG3.
A14. The chimeric molecule of any one of embodiments A1 to A13, wherein the CD4 co-receptor protein is a human CD4 co-receptor protein, the LAG3 co-receptor protein is a human LAG3 co-receptor protein and the CD8 co-receptor protein is a human CD8 co-receptor protein.
A15. The chimeric molecule of any one of embodiments A1 to A14, wherein the peptide is attached to the N-terminus of the co-receptor via a linker.
A16. The chimeric molecule of embodiment A15, wherein the linker has a length between 5 and 30 amino acids.
A17. The chimeric molecule of embodiments A15 or A16, wherein at least 40%, at least 50%, at least 60%, at least 70%, at least 80%, at least 90% of the amino acid residues in the linker are glycine or serine residues.
A18. The chimeric molecule of any one of embodiments A15 to A17, wherein the linker comprises the amino acid sequence (GGGGS)ₓ, wherein G is glycine, S is serine and x is the number of repetitions, wherein x can be any number between 1 and 5.
A19. A polynucleotide encoding the chimeric molecule of any one of embodiments A1 to A18.
A20. A library of polynucleotides comprising a plurality of polynucleotides of embodiment A19.
A21. The library of polynucleotides of embodiment A20, wherein at least two polynucleotides of the library encode an identical co-receptor protein attached to a different peptide.
A22. A cell comprising the polynucleotide of embodiment A9.
A23. A method for simultaneously identifying antigen-specific T cell receptors and the peptides specifically recognized by said T cell receptors (TCRs), the method comprising the steps of:
   (a) providing polyclonal T cells of interest expressing the library of polynucleotides of embodiments A20 or A21;
   (b) contacting the T cells of step (a) with antigen presenting cells (APC) comprising a major histocompatibility complex (MHC);
   (c) isolating at least one T cell that is activated upon contacting with the APCs in step (b);
   (d) sequencing the DNA of the isolated T cells of step (c) to obtain information about the TCR sequences and the peptide sequences attached to the CD4, LAG-3 or CD8 co-receptors present in these T cells; and
   (e) identifying cognate T cell receptor-peptide pairs based on the sequencing data obtained in step (d).
A24. A method for identifying at least one antigen-specific T cell receptor, the method comprising the steps of:
   (a) providing polyclonal T-cells of interest expressing a polynucleotide of embodiment A19;
   (b) contacting the T-cells of step (a) with antigen presenting cells (APC) comprising a major histocompatibility complex (MHC);
   (c) isolating at least one T-cell that is activated upon contacting with the APCs in step (b);
   (d) sequencing of the TCR loci of the at least one T cell isolated in step (c); and
   (e) identifying at least one T cell receptor encoded by the TCR loci of the at least one T cell to be antigen-specific.
A25. A method for identifying at least one T cell-specific antigen, the method comprising the steps of:
   (a) providing monoclonal T-cells of interest expressing a polynucleotide of embodiment A19 or a library of polynucleotides of embodiments A20 or A21;
   (b) contacting the T cells of step (a) with antigen presenting cells (APC) comprising a major histocompatibility complex (MHC);
   (c) isolating at least one T cell that is activated upon contacting with the APCs in step (b);
   (d) sequencing the part of the polynucleotide encoding the peptide attached to the N-terminus of a CD4, LAG-3 or CD8 co-receptor protein of the at least one T cell isolated in step (c); and
   (e) identifying at least one peptide encoded by the polynucleotide comprised in the at least one T cell to be a T cell-specific antigen.
A26. The method of any one of embodiments A23 to A25, wherein the APC is an autologous or a heterologous ARC.
A27. The method of any one of embodiments A23 to A26, wherein the APC is a genetically modified autologous or heterologous cell or cell line, expressing a mutated MHC molecule.
A28. The method of embodiment A27, wherein the mutated MHC molecule is a MHC class II molecule comprising the extracellular MHC class II alpha chain and a native or heterologous transmembrane domain, as well as the extracellular MHC class II beta chain and a native or heterologous transmembrane domain.
A29. The method of embodiment A27, wherein the mutated MHC molecule is a MHC class I molecule comprising the extracellular MHC class I alpha chain and a native or heterologous transmembrane domain, as well as beta-2 microglobulin.
A30. The method of embodiments A23 to A29, wherein the co-receptor protein encoded by the polynucleotide or the library of polynucleotides is CD8 if the MHC molecule comprised in the APC is a MHC class I molecule.
A31. The method of embodiments A23 to A29, wherein the co-receptor protein encoded by the polynucleotide is CD4 or LAG-3 if the MHC molecule expressed by the APC is a MHC class II molecule.
A32. A method for treating a subject suffering fro cancer, the method comprising the steps of:
   (a) Identifying at least one antigen-specific T cell receptor and/or at least one T cell-specific antigen with the methods of embodiments A23 to A31;
   (b) administering to the subject suffering from cancer the at least one T cell receptor and/or T cell-specific antigen identified in step (a).
A33. The method of embodiment A32, wherein the antigen-specific T cell receptor is administered to the subject by virus-mediated gene delivery.
A34. The method of embodiment A32, wherein the T cell-specific antigen is administered to the subject in form of a peptide or in form of a polynucleotide encoding a peptide.
A35. The method of embodiment A34, wherein the peptide or the polynucleotide encoding the peptide is attached to a compound that improves delivery of the peptide or polynucleotide encoding the peptide to an APC.

The present invention provides for the first time a method which allows simultaneous detection and enrichment of antigen-specific T cells and identification of the TCRs and their cognate epitopes.

Accordingly, in one embodiment, the invention relates to a chimeric molecule comprising the CD4, LAG3 or CD8 co-receptor protein and a peptide attached to the N-terminus of the co-receptor.

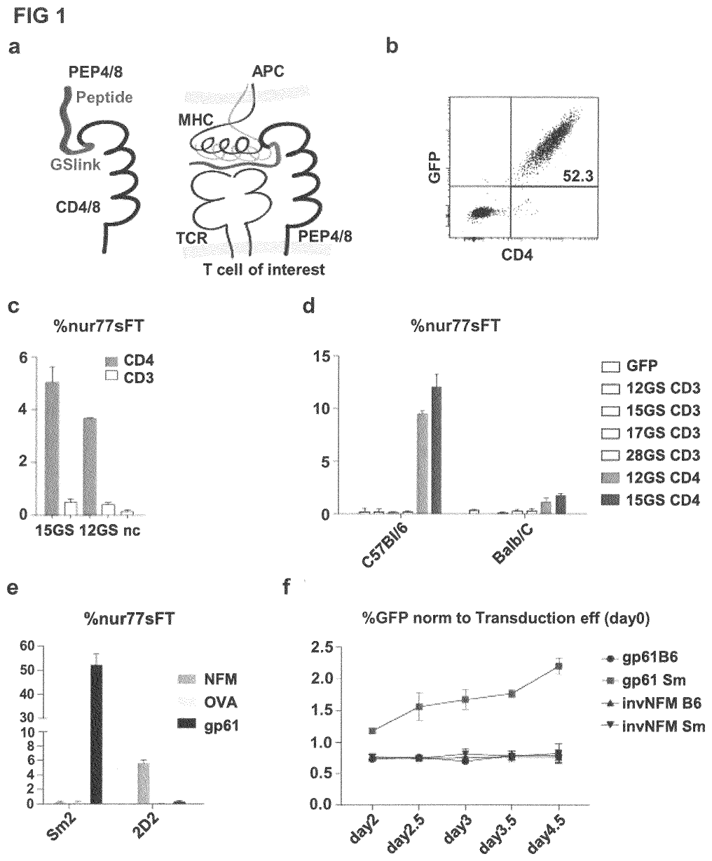

That is, the present invention relates to a chimeric molecule that facilitates the identification of peptides that can be presented by an MHC molecule such that the peptide is recognized by a T cell receptor. It has been surprisingly found that expressing a polynucleotide encoding the chimeric molecule of the invention in a T cell allows the formation of a complex between the peptide that is covalently attached to the N-terminus of the co-receptor and a major histocompatibility complex on the surface of an antigen presenting cell (APC) that is located in close proximity to the T cell. This peptide-MHC complex may then be recognized by the T cell receptor of the same T cell, resulting in activation of this T cell (see FIG. 1A). Accordingly, both the T cell receptor and its cognate antigenic peptide, which is part of the chimeric molecule of the present invention, are encoded by the same T cell, thereby significantly facilitating the identification of cognate TCR-antigen pairs compared to previous methods. Even though both the TCR and the antigenic peptide are present on the surface of the same T cell, the T cell can only be activated by the antigenic peptide, if the antigenic peptide has undergone formation of a complex with a suitable MHC molecule on the surface of other cells.

It has been demonstrated in the examples that T cells can get activated if a cognate antigenic peptide is attached to the N-terminus of the co-receptor CD4 (FIG. 1C-E). In contrast, no activation was observed when a non-cognate antigenic peptide was attached to the co-receptor protein. In addition, no activation of the T cell was observed when a cognate antigenic peptide was attached to the CD3, indicating that the attachment of an antigenic peptide to the N-terminus of a co-receptor that directly interacts with MHC molecules, such as the co-receptor CD4, is required for the formation of a peptide-MHC complex. Thus, in a particular embodiment, the invention relates to the chimeric molecule of the invention, wherein the co-receptor protein is CD4.

The chimeric molecules of the present invention differ significantly from previous fusion proteins comprising certain parts of CD4. Al-Jaufy et al. (Infect Immun. 1995 August; 63(8): 3073-3078.), for example, fused the Shiga toxin A subunit to the 180 N-terminal amino acids of CD4 (433 amino acids in total), which retain the capacity to bind to glycoprotein 120 on the surface of HIV, to generate a cytotoxic fusion protein for the treatment of HIV. Breuer et al. (PLoS One. 2011; 6(5): e20033.) designed a synthetic protein inhibitor against the HIV-1 pathogenicity factor Nef which comprises a 37 amino acid motif of C04. Thus, both prior art documents relate to soluble proteins that only comprise a short fragment of CD4. The embodiments of the present invention, on the other hand, are related to co-receptor proteins, which are understand by the person skilled in the art to be cell surface receptors that are anchored to the cell membrane.

The co-receptors CD4 and LAG3 share approximately 20% sequence identity in humans and both bind to MHC class II molecules to facilitate the recognition of the peptide-MHC class II complex by a T cell receptor. The co-receptor CD8 fulfils a similar role in the interaction between a T cell receptor and a peptide-MHC complex comprising an MHC class I molecule. Thus, it is plausible that the methods of the present invention can also be carried out with a chimeric molecule comprising the co-receptors LAG3 or CD8.

The peptide that is attached to the co-receptor protein may be any peptide. As discussed above, the peptide that is attached to the co-receptor protein is preferably a peptide that has the potential to be presented by an MHC molecule. A peptide is said to have the potential to be presented by a major histocompatibility complex, if the peptide and the MHC form a peptide-MHC complex that can be recognized by a T cell receptor, wherein the T cell receptor may be any T cell receptor. As used herein, the term “peptide-MHC complex” refers to an MHC molecule (MHC class I or MHC class II) with a peptide bound in the art-recognized peptide binding pocket of the MHC. Within the present invention, the entire peptide that is attached to the co-receptor protein may be involved in the formation of the peptide-MHC complex. However, the invention also encompasses peptides, wherein only a part of the peptide is involved in the formation of the peptide-MHC complex. Accordingly, in one embodiment, the invention relates to a chimeric molecule comprising the CD4, LAG3 or CD8 co-receptor protein and a peptide attached to the N-terminus of the co-receptor, wherein the peptide or a part of the peptide can be presented by a major histocompatibility complex.

In a particular embodiment, the invention relates to the chimeric molecule of the invention, wherein the peptide has a length of 6 to 200 amino acid residues.

That is, the peptide that is attached to the co-receptor protein may be a peptide with a length of 6 to 200 amino acid residues. In particular, it is preferred that at least a part of this peptide can be involved in the formation of a peptide-MHC complex.

Peptides that are displayed by MHC class I molecules, also herein referred to as MHC class I epitopes or MHC class I peptides, typically have a length of 8 to 15 amino acids, with the majority of peptides having a length of 9 amino acids. Peptides that are displayed by MHC class II molecules, also herein referred to as MHC class II epitopes or MHC class II peptides typically have a length of 11 to 30 amino acids. Thus, in a particular embodiment, the invention relates to the chimeric molecule of the invention, wherein the peptide has a length of 7 to 30 amino acid residues.

The peptides that are attached to the co-receptor protein may comprise further amino acids attached to the N- and/or C-terminus of an MHC class I or II epitope without significantly affecting the binding of the MHC class I or II epitope to an MHC molecule. Thus, in an alternative embodiment, the invention relates to the chimeric molecule of the invention, wherein the peptide has a length of 30 to 50 amino acid residues.

In a particular embodiment, the invention relates to the chimeric molecule of the invention, wherein the peptide is a random peptide.

That is, the peptide that is attached to the co-receptor protein may have any amino acid sequence. The chimeric molecule of the invention may be used for the identification of peptides that can stimulate a T cell receptor when bound by an MHC molecule. A “random peptide” as used herein may be a peptide with a random amino acid sequence that does not share any sequence identity with peptides that are known to be bound by MHC class I or MHC class II molecules.

Alternatively, the peptide that is attached to the co-receptor protein may share sequence identity with peptides or proteins that have been previously described. Thus, in a particular embodiment, the invention relates to the chimeric molecule of the invention, wherein the peptide is a peptide that is encoded by a given DNA or cDNA molecule.

That is, the chimeric molecule of the invention may be used for the identification of previously unknown antigenic peptides. For example, a DNA or cDNA molecule encoding a peptide may be cloned into a polynucleotide encoding a co-receptor protein such that a polynucleotide encoding the chimeric molecule of the invention is obtained. It may then be tested with the methods of the invention if the peptide that is attached to the co-receptor protein can undergo the formation of a peptide-MHC complex such that the T cell that expresses the polynucleotide encoding the chimeric molecule is activated.

The DNA or cDNA molecule may be obtained from any source. In particular, the DNA may be obtained from an antigen-presenting cell or the cDNA may be obtained by reverse transcription of RNA obtained from an antigen presenting cell. The antigen presenting cell may be a tumor cell that has been obtained in a biopsy. Alternatively, the antigen presenting cell may be a cell that has been infected with a pathogen. Alternatively, the DNA or cDNA molecule may be a DNA or cDNA molecule encoding a peptide or protein that is to be administered to a subject. The development of drugs or other peptides or proteins that are administered to subjects usually involves immunogenicity testing to ensure that administering the compound to a subject does not result in unwanted immunogenic reactions. Accordingly, the DNA or cDNA molecule encoding a peptide or protein, or a fraction of said DNA or cDNA molecule, may be cloned into a polynucleotide encoding a co-receptor protein to obtain a polynucleotide encoding the chimeric molecule of the invention.

Alternatively, the DNA or cDNA molecule may be obtained directly from a pathogen and cloned into a polynucleotide encoding a co-receptor protein to obtain a polynucleotide encoding the chimeric molecule of the invention. A pathogen, as used herein, preferably refers to a viral or bacterial pathogen

The term “given DNA or cDNA molecule” refers to any DNA or cDNA molecule that has been directly or indirectly obtained from a cell or pathogen. For example, a DNA molecule may be directly obtained from a cell or pathogen through isolation of DNA from said cell or pathogen. A cDNA molecule may be directly obtained from a cell or pathogen through isolation of RNA and reverse transcription of the RNA into cDNA. However, DNA molecules may also be indirectly obtained, for example through chemical synthesis of computationally designed polynucleotide sequences. The computationally designed polynucleotide sequences may be based, for example on exome sequencing data from single cells or from peptide sequencing data, in particular from sequencing of peptides that have been isolated from the surface of antigen presenting cells. Alternatively, computationally designed polynucleotide sequences may based on genomic data from pathogens.

The DNA or cDNA molecule may be obtained by any method known in the art. In a particular embodiment, the method relates to the chimeric molecule of the invention, wherein the DNA or cDNA molecule encoding the peptide is obtained by fragmentation of a larger DNA or cDNA molecule.

That is, the peptide may be encoded by a DNA or cDNA molecule that has been obtained by fragmentation of a larger DNA or cDNA molecule.

Fragmentation of a DNA or cDNA molecule may be achieved in a targeted or untargeted manner, for example by using endonucleases. However, fragmentation of DNA or cDNA molecules may also be achieved by random shearing of these molecules. Alternatively, a fragment of a known DNA or cDNA molecule may also be obtained by methods of molecular cloning, for example by PCR. The skilled person is aware of methods of combining a DNA or cDNA fragment with a polynucleotide encoding a co-receptor protein such that a polynucleotide encoding the chimeric molecule of the invention is obtained.

Thus, in a particular embodiment, the invention relates to the chimeric molecule of the invention, wherein the peptide is derived from a tumor cell or from a cell that has been infected with a pathogen.

The chimeric molecule of the invention may be used for the identification of previously unknown tumor or pathogen-associated antigens. A peptide is said to be derived from a tumor cell, if the peptide comprises an amino acid sequence that shares sequence identity with a peptide or protein that is synthesized in a tumor cell. A peptide is said to be derived from a cell that has been infected with a pathogen, if the peptide comprises an amino acid sequence that shares sequence identity with a peptide or protein that is synthesized in a cell that has been infected with a pathogen. The peptide that is derived from a tumor cell or a cell that has been infected with a pathogen may share sequence identity with any peptide or fraction of a protein that can be found in these cells.

In certain embodiments, the peptide that is attached to the co-receptor protein of the invention may comprise the amino acid sequence of a peptide that has been previously described to be presented by an MHC I or MHC II molecule. A chimeric molecule comprising a peptide that is known to be presented by an MHC class I or II molecule may allow identifying T cell receptors that are efficiently stimulated by this peptide. The peptide that is known to be presented by an MHC class I or II peptide may be a peptide that is derived from a tumor cell or from a cell that has been infected with a pathogen.

In certain embodiments, the chimeric molecule of the invention is used for the identification of tumor-specific T cell receptors. For example, a peptide that is known to be presented on the surface of a subject's tumor cells by an MHC molecule may be attached to a co-receptor protein to identify T cell receptors that specifically recognize this epitope. Thus, in a particular embodiment, the invention relates to the chimeric molecule of the invention, wherein the peptide comprises an epitope of a tumor antigen. A peptide is said to comprise an epitope of a tumor antigen, if the peptide comprises an amino acid sequence that is identical to the amino acid sequence of an epitope of a tumor antigen.

The term “tumor antigen” as used herein, can be a tumor-associated antigen or a tumor-specific antigen, and indicates a molecule (e.g., a protein or peptide) that is expressed by a tumor cell and either (a) differs qualitatively from its counterpart expressed in normal cells, or (b) is expressed at a higher level in tumor cells than in normal cells. Thus, a tumor antigen can differ from (e.g., by one or more amino acid residues where the molecule is a protein) or it can be identical to its counterpart expressed in normal cells. Some tumor antigens are not expressed by normal cells, or are expressed at a level at least about two-fold higher (e.g., about two-fold, three-fold, five-fold, ten-fold, 20-fold, 40-fold, 100-fold, 500-fold, 1,000-fold, 5,000-fold, or 15,000-fold higher) in a tumor cell than in the tumor cell's normal counterpart.

In certain embodiments, a tumor antigen is an immunogenic protein expressed in or on a neoplastic cell or tumor, such as a cancer cell or malignant tumor. In certain embodiments, a tumor antigen is a non-specific, mutant, overexpressed or abnormally expressed protein, which can be present on both a neoplastic cell or tumor and a normal cell or tissue. In certain embodiments, a tumor antigen is a tumor-specific antigen which is restricted to tumor cells. In certain embodiments, a tumor antigen is a cancer-specific antigen which is restricted to cancer cells.

In certain embodiments, a tumor antigen can exhibit one, two, three, or more, including all, of the following characteristics: overexpressed/accumulated (i.e., expressed by both normal and neoplastic tissue, but highly expressed in neoplasia), oncofetal (i.e., usually only expressed in fetal tissues and in cancerous somatic cells), oncoviral or oncogenic viral (i.e., encoded by tumorigenic transforming viruses), cancer-testis (i.e., expressed only by cancer cells and adult reproductive tissues, e.g., the testis), lineage-restricted (i.e., expressed largely by a single cancer histotype), mutated (i.e., only expressed in neoplastic tissue as a result of genetic mutation or alteration in transcription), post-translationally altered (e.g., tumor-associated alterations in glycosylation), or idiotypic (i.e., developed from malignant clonal expansions of B or T lymphocytes).

In certain embodiments, the tumor antigen includes antigens from neoplastic diseases including acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In certain embodiments, the tumor antigen includes oncogenic viral antigens, cancer-testis antigens, oncofetal antigens, tissue differentiation antigens, mutant protein antigens, neoantigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUC1, MUC5AC, p53 (non-mutant), PAX5, PBF, PRAME, PSMA, RAGE, RAGE-1, RGS5, RhoC, RNF43, RU2AS, secernin 1, SOXIO, STEAP1 (six-transmembrane epithelial antigen of the prostate 1), survivin, Telomerase, VEGF, WT1, EGF-R, CEA, CD20, CD33, CD52, glycoprotein 100 (GP100 or gp 100 protein), MEL ANA/MART 1, MART2, NY-ESO-I, p53, MAGE AI, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDK 2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPR, H-ras, K-ras (V-Ki-ras2 Kirsten rat sarcoma viral oncogene), N-ras, RBAF600, SIRT2, SNRPD1, SSX, SSX2, SYT-SSX1 or-SSX2 fusion protein, TGF-betaRll, Triosephosphate isomerase, ormdm-2, LMP2, HPV E6/E7, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin BI, Polysialic acid, MYCN, TRP2, TRP2-Int2, GD3, Fucosyl GM1, Mesothelin, PSCA, sLe(a), cypIBI, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP1, CA-125, CA19-9, Calretinin, Epithelial membrane antigen (EMA), Epithelial tumor antigen (ETA), CD 19, CD34, CD99, CD117, Chromogranin, Cytokeratin, Desmin, Glial fibrillary acidic protein (GFAP), gross cystic disease fluid protein (GCDFP-15), HMB-45 antigen, Myo-DI, muscle-specific actin (MSA), neurofilament, neuron-specific enolase (NSE), placental alkaline phosphatase, synaptophysis, thyro globulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-A11, HSP70-2, KIAA0205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F,5T4, 791Tgp72, 13HCG, BCA225, BTAA, CD68\KP1, CO-029, HTgp-175, M344, MG7-Ag, MOV18, NB\70K, NY-CO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and RORI.

In certain embodiments, the tumor antigen includes oncogenic viral antigens, wherein the oncogenic virus antigens are antigens of human papillomavirus (HPV), antigens of Kaposi's sarcoma-associated herpesvirus, such as latency-associated nuclear antigen, antigens of Epstein-Barr virus, such as EBV-EA, EBV-MA, or EBV-VCA, antigens of Merkel cell polyomavirus, such as MCV T antigen, or antigens of human T-lymphotropic virus, such as HTLV-1 Tax antigen.

In certain embodiments, the tumor antigen is a tumor-associated antigen or a tumor-specific antigen.

In certain embodiments, the tumor antigen is a neoantigen. A “neoantigen,” as used herein, means an antigen that arises by mutation in a tumor cell and such an antigen is not generally expressed in normal cells or tissue. Without being bound by theory, because healthy tissues generally do not posses these antigens, neoantigens represent a preferred target. Additionally, without being bound by theory, in the context of the present invention, since the T cells that recognize the neoantigen may not have undergone negative thymic selection, such cells can have high avidity to the antigen and mount a strong immune response against tumors, while lacking the risk to induce destruction of normal tissue and autoimmune damage. Thus, in a particular embodiment, the invention relates to the chimeric molecule of the invention, wherein the tumor antigen is a neoantigen.

In certain embodiments, the tumor antigen can be an antigen ortholog, e.g., a mammalian (i.e., non-human primate, pig, dog, cat, or horse) to a human tumor antigen.

The term “epitope” as used herein refers to that portion of an antigen that makes contact with a particular immunoglobulin, such as a T cell receptor.

The chimeric molecule of the invention may also be used to identify mutated variants of epitopes of known tumor antigens that are efficiently recognized by a T cell receptor. Thus, in a particular embodiment, the invention relates to the chimeric molecule of the invention, wherein the peptide comprises an amino acid sequence with at least 50%, 60%, 70%, 80%, 90% sequence identity with an epitope of a tumor antigen.

That is, the chimeric molecule of the invention may be used to identify mutated antigenic peptides that are recognized by an MHC molecule and result in more or less efficient activation of a T cell compared to their unmutated counterparts. Within the present invention, it is preferred that the chimeric molecules are encoded by a polynucleotide that is expressed in a cell. The skilled person is aware of methods to introduce mutations into DNA sequences encoding a peptide either randomly, or in a targeted fashion, such that a mutated peptide is obtained.

In a particular embodiment, the invention relates to the chimeric molecule of the invention, wherein the peptide comprises an MHC class I epitope when the co-receptor protein is CD8.

That is, the co-receptor CD8 facilitates the binding of a T cell receptor to a peptide-MHC class I complex. Thus, it is preferred that the peptide that is attached to the co-receptor CD8 comprises an MHC class I epitope.

In a particular embodiment, the invention relates to the chimeric molecule of the invention, wherein the epitope is an MHC class II epitope when the co-receptor proteins is CD4 or LAG3.

That is, the co-receptors CD4 and LAG3 facilitate the binding of a T cell receptor to a peptide-MHC class II complex. Thus, it is preferred that the peptide that is attached to the co-receptors CD4 or LAG3 comprises an MHC class II epitope.

In cases where a peptide is yet to be identified to be presented by an MHC molecule, the peptide may be attached to any co-receptor, i.e. CD4, CD8 or LAG3.

The co-receptor proteins of the chimeric molecule may be derived from any mammal. However, it is preferred that the co-receptor is derived from the same organism as the T cell that it is intended to be synthesized in. For example, if it is intended to identify a cognate antigenic peptide—T cell receptor pair in a human, it is preferred that the chimeric molecule comprises a human co-receptor protein. The CD4, LAG-3 or CD8 co-receptor proteins are in particular full length CD4, LAG-3 or CD8 co-receptor proteins.

Within the present invention, it is preferred that the co-receptor protein is a human co-receptor protein. Thus, in a particular embodiment, the invention relates to the chimeric molecule of the invention, wherein the CD4 co-receptor protein is a human CD4 co-receptor protein, the LAG3 co-receptor protein is a human LAG3 co-receptor protein or the CD8 co-receptor protein is a human CD8 co-receptor protein.

That is, in certain embodiments, the chimeric molecule comprises a human CD4 co-receptor protein, wherein the peptide is attached to the N-terminus of CD4. In other embodiments, the chimeric molecule comprises a human LAG3 co-receptor protein, wherein the peptide is attached to the N-terminus of LAG3. In other embodiments the chimeric molecule comprises a human CD8 co-receptor protein, wherein the peptide is attached to the N-terminus of the CD8-alpha chain or the CD8-beta chain. In case the peptide is attached to the N-terminus of the CD8-beta chain, it is preferred that the cell also synthesizes a non-modified CD8-alpha chain. Accordingly, the chimeric molecules comprising the human CD4, CD8 or LAG3 co-receptor proteins are preferably synthesized in human cells, in particular, human T cells.

In a particular embodiment, the invention relates to a chimeric molecule of the invention, wherein the peptide is attached to the N-terminus of the co-receptor via a linker.

That is, the chimeric molecule may comprise a linker between the co-receptor and the peptide to optimize the recognition between the peptide, the T cell receptor and the MHC molecule on the APC.

The linker may have any length that allows efficient interaction of the peptide with the TCR and the MHC molecule on the APC. In particular, the linker may have a length between 5 and 30 amino acid residues. Thus, in a particular embodiment, the invention relates to a chimeric molecule, wherein the linker has a length between 5 and 30 amino acids.

To allow the peptide to efficiently interact with the TCR and the MHC molecule on the APC, it is preferred that the linker is a flexible linker. The skilled person is aware of linkers with a high degree of flexibility. For example, linkers with a high percentage of glycine or serine residues are known to have a high degree of flexibility. Thus, in a particular embodiment, the invention relates to a chimeric molecule of the invention, wherein at least 40%, at least 50%, at least 60%, at least 70%, at least 80%, at least 90% of the amino acid residues in the linker are glycine or serine residues.

The linker sequence GGGGS, consisting of four glycine residues and a C-terminal serine residue is frequently used in the art as a flexible peptide linker to tether a first polypeptide to a second polypeptide. Thus, the linker of the invention preferably comprises one or more motifs with the sequence GGGGS. Accordingly, in a particular embodiment, the invention relates to a chimeric molecule of the invention, wherein the linker comprises the amino acid sequence (GGGGS)ₓ, wherein G is glycine, S is serine and x is the number of repetitions, wherein x can be any number between 1 and 5.

Thus, in certain embodiments, the linker comprises the amino acid sequence GGGGS once. In other embodiments, the linker comprises the amino acid sequence GGGGS twice. In other embodiments, the linker comprises the amino acid sequence GGGGS three times. In other embodiments, the linker comprises the amino acid sequence GGGGS four times. In other embodiments, the linker comprises the amino acid sequence GGGGS five times. If the linker comprises the amino acid sequence GGGGS more than once, the amino GGGGS sequences may be contiguous or may be interrupted by one or more other amino acids.

It is shown in the examples that the highest activation of T cells can be observed if the peptide is attached to the co-receptor with a linker consisting of 12 or 15 amino acids (FIG. 1 C and D). Thus, in a preferred embodiments, the invention relates to the chimeric molecule of the invention, wherein the linker has a length of 12 to 15 amino acid residues. In a more preferred embodiment, the invention relates to the chimeric molecule of the invention, wherein the linker comprises the sequence GSGGGGSGGGGS (SEQ ID NO. 1). In an even more preferred embodiment, the invention relates to the chimeric molecule of the invention, wherein the linker comprises the sequence GSGGSGGGGSGGGGS (SEQ ID NO. 2).

In a particular embodiment, the invention relates to a polynucleotide encoding a chimeric molecule of the invention.

Within the present invention, it is intended that the chimeric molecule of the invention is synthesized in a cell through expression of a polynucleotide encoding said chimeric molecule. In addition, the polynucleotide encoding the chimeric molecule of the invention may further comprise a polynucleotide encoding a signal peptide that directs the chimeric molecule to the cell surface.

The term “polynucleotide” as used herein refers to a sequence of nucleotides connected by phosphodiester linkages. A polynucleotide of this invention can be a deoxyribonucleic acid (DNA) molecule or ribonucleic acid (RNA) molecule in either single- or double-stranded form. Nucleotide bases are indicated herein by a single letter code: adenine (A), guanine (G), thymine (T), cytosine (C), inosine (I) and uracil (U). A polynucleotide of this invention can be prepared using standard techniques well known to one of ordinary skill in the art. This term is not to be construed as limiting with respect to the length of a polymer, and encompasses known analogues of natural nucleotides, as well as nucleotides that are modified in the sugar and/or phosphate moieties. This term also encompasses nucleic acids containing modified backbone residues or linkages, which may be synthetic or naturally-occurring, and which have similar binding properties as the reference nucleic acid and are metabolized in a manner similar to the reference nucleotides.

In a particular embodiment, the invention relates to a library of polynucleotides comprising a plurality of polynucleotides of the invention.

The invention further encompasses a library of polynucleotides. The library of polynucleotides of the invention comprises a plurality of polynucleotides that encode chimeric molecules of the invention. For example, the library of polynucleotides may comprise two or more polynucleotides that encode a chimeric molecule of the invention. In certain embodiments, the library of polynucleotides may comprise at least 100, at least 1,000, at least 10,000, at least 100,000, at least 1,000,000 or at least 10,000,000 polynucleotides that encode a chimeric molecule of the invention.

In certain embodiments, the polynucleotides in the library of polynucleotides are comprised in a larger polynucleotide. In certain embodiments, the polynucleotides in the library of polynucleotides are comprised in circular polynucleotides, such as DNA or RNA vectors. In certain embodiments, the polynucleotides in the library of polynucleotides are comprised in viral vectors that can be used for virus-mediated gene delivery.

In a particular embodiment, the invention relates to the library of polynucleotides of the invention, wherein at least two polynucleotides of the library encode an identical co-receptor protein attached to a different peptide.

That is, the library of polynucleotides of the invention may be used for the identification of antigenic peptides that can induce a T cell response. It is preferred that the polynucleotides of the library encode an identical co-receptor protein but differ in the region of the polynucleotide that encodes the peptide. That is, in certain embodiments, the library of the invention comprises at least two polynucleotides, wherein each of these polynucleotides encodes the co-receptor CD4 protein attached to a peptide with a different amino acid sequence. In other embodiments, the library of the invention comprises at least two polynucleotides, wherein each of these polynucleotides encodes the co-receptor protein CD8 attached to a peptide with a different amino acid sequence. In other embodiments, the library of the invention comprises at least two polynucleotides, wherein each polynucleotide encodes the co-receptor protein LAGS attached to a peptide with a different amino acid sequence.

The peptides that are encoded in the library of polynucleotides of the invention may have any amino acid sequence. For example, the peptides may be have sequence identity with a known antigenic peptide, for example an antigenic peptide derived from a tumor cell or from a cell that has been infected by a pathogen. In this case, the library of polynucleotides may be obtained by introducing random or targeted mutations into a polynucleotide encoding a co-receptor protein and a peptide comprising an amino acid sequence of a known antigenic peptide. In particular, the mutations may be exclusively introduced into the part of the polynucleotide that encodes the antigenic peptide.

In certain embodiments, the library of polynucleotides of the invention may be used to identify previously unknown antigenic peptides. For example, a library of polynucleotides may be generated by isolating mRNAs from a cell, in particular an antigen presenting cell, and reverse transcribing the mRNAs into cDNA. The cDNA may then be cloned into a plurality of polynucleotides encoding a co-receptor protein to obtain a library of polynucleotides of the invention. In particular the cDNAs may be fragmented before the cloning step and the resulting fragments may be cloned into the plurality of polynucleotides encoding the co-receptor protein. In certain embodiments, mRNA is isolated from a tumor cell or a cell that has been infected with a pathogen. The skilled person is aware of methods for RNA isolation, reverse transcription and molecular cloning.

In other embodiments, the library of polynucleotides may be generated based on exome sequencing data. The skilled person is aware of methods for sequencing the exome of a cell. The exome data may be obtained from any cell. In certain embodiments, the exome data is obtained from a tumor cell or from a cell that has been infected with a pathogen. The obtained exome data may then be processed by bioinformatic methods to predict a multitude of DNA fragments of predetermined size that cover parts or, preferably, the entire exome of a cell. The predicted DNA fragments may have any size. However, it is preferred that the predicted DNA fragments have a size of 30 to 300 base pairs, more preferably of 60 to 150 base pairs, most preferably of 75 to 105 base pairs. The predicted DNA fragments may then be chemically synthesized and cloned into a plurality of polypeptides encoding a co-receptor to obtain the library of polynucleotides of the invention.

In certain embodiments, peptides that are presented on the surface of an APC may be isolated from the APC and their sequence may be determined by any method known in the art, in particular by mass spectrometry. Polynucleotides encoding these isolated peptides may then be generated, for example by chemical synthesis, and cloned into a plurality of polynucleotides encoding a co-receptor protein to generate the library of polypeptides of the invention. Such a library may, for example, be used to identify which of the peptides that have been isolated from the cell surface can be recognized by a T cell receptor in general and, specifically, by which T cell receptor.

As mentioned above, the library of polynucleotides preferably comprises at least two polynucleotides that encode an identical co-receptor protein attached to a different peptide. However, it is even more preferred that at least 10, at least 50, at least 100, at least 500, at least 1,000, at least 10,000, at least 100,000, or at least 1,000,000 of the polynucleotides of the library encode an identical co-receptor protein, but differ in the sequence of the peptide attached to the co-receptor protein.

The skilled person is aware of methods to generate a library of polynucleotides according to the invention. For example, the library according to the invention may be generated by molecular cloning. For example, a plurality of identical DNA vectors comprising a polynucleotide encoding a co-receptor protein and, optionally a linker and a signal peptide may first be digested with one or more restriction enzymes. In a second step, a plurality of polynucleotides encoding peptides with different amino acid sequences may be digested with compatible restriction enzymes and ligated into the vector encoding the co-receptor protein. Preferably, the polynucleotide encoding the peptide is introduced into the vector upstream of the polynucleotides encoding the co-receptor protein and the linker and downstream of the polynucleotide encoding the signal peptide such that the peptide will be attached to the N-terminus of co-receptor protein. Alternatively, the polynucleotides that are comprised in the library of the invention may be designed computationally and synthesized by chemical DNA synthesis.

In a particular embodiment, the invention relates to a cell comprising the polynucleotide of the invention.

That is, the invention encompasses any cell comprising a polynucleotide of the invention. Preferably, the cell comprising the polynucleotide of the invention is a T cell.

In a particular embodiment, the invention relates to a method for simultaneously identifying antigen-specific T cell receptors and the peptides specifically recognized by said T cell receptors (TCRs), the method comprising the steps of: (a) providing polyclonal T-cells of interest expressing the library of polynucleotides of the invention; (b) contacting the T-cells of step (a) with antigen presenting cells (APC) comprising a major histocompatibility complex (MHC); (c) isolating at least one T-cell that is activated upon contacting with the APCs in step (b); (d) sequencing the DNA of the isolated T cells of step (c) to obtain information about the TCR sequences and the peptide sequences attached to the CD4, LAG-3 or CD8 co-receptors present in these T cells; and (e) identifying cognate T cell receptor-peptide pairs based on the sequencing data obtained in step (d).

That is, the method of the invention may be used for the simultaneous identification of cognate T cell-receptor-peptide pairs. Previous methods for the identification of cognate TCR-peptide pairs have the disadvantage that they only allow screening of multiple T cell receptors against a single antigenic peptide at a time, thereby resulting in time and cost intensive screening approaches. The method of the present invention, on the other hand, has the advantage that the cells expressing a polynucleotide encoding a chimeric molecule of the invention comprise the genetic information of the T cell receptor and the antigenic peptide that stimulates the T cell receptor. Thus, the method of the invention allows screening a polyclonal T cell population against a plurality of peptides in a single experiment and to enrich and identify the T cells that synthesize cognate TCR-peptide pairs. Accordingly, the method of the invention can significantly facilitate the identification of previously unknown antigenic peptides and of the T cell receptors that recognize these peptides.

For that, in a first step, a library of polynucleotides encoding the chimeric molecule of the invention is introduced into a population of polyclonal T cells. The skilled person is aware of methods for introducing polynucleotides into T cells. For example, a polynucleotide may be introduced into a T cell by transfection or viral transduction. It is intended that the T cells in the population of T cells are transfected or transduced with the library of polynucleotides such that each T cell does not obtain more than one polynucleotide encoding the chimeric molecule of the invention. Polyclonal T cells are said to express the library of polynucleotides of the invention, if the T cells produce detectable amounts of the chimeric molecule of the invention. Preferably, at least two of the T cells in the polyclonal population of T cells express a polynucleotide encoding an identical co-receptor protein attached to a peptide with a different amino acid sequence.

The term “population” as used herein refers to more than one cell of the same cell type. A “population of polyclonal T cells”, as used herein, refers to a population of T cells, wherein at least two of the T cells comprised in the population comprise a different T cell receptor. The population of polyclonal T cells may be of any source. For example, the population of polyclonal T cell may be obtained from a subject. The skilled person is aware of methods to isolate T cells from a sample that has been obtained from a subject, such as blood, spleen, lymph nodes or tumor tissue or from any other suitable source. The present invention also encompasses populations of polyclonal T cells that have been obtained by genetic engineering. For example, a population of TCR negative cells may be transfected or transduced with a library encoding a plurality of T cell receptors such that a population of polyclonal T cells is obtained.

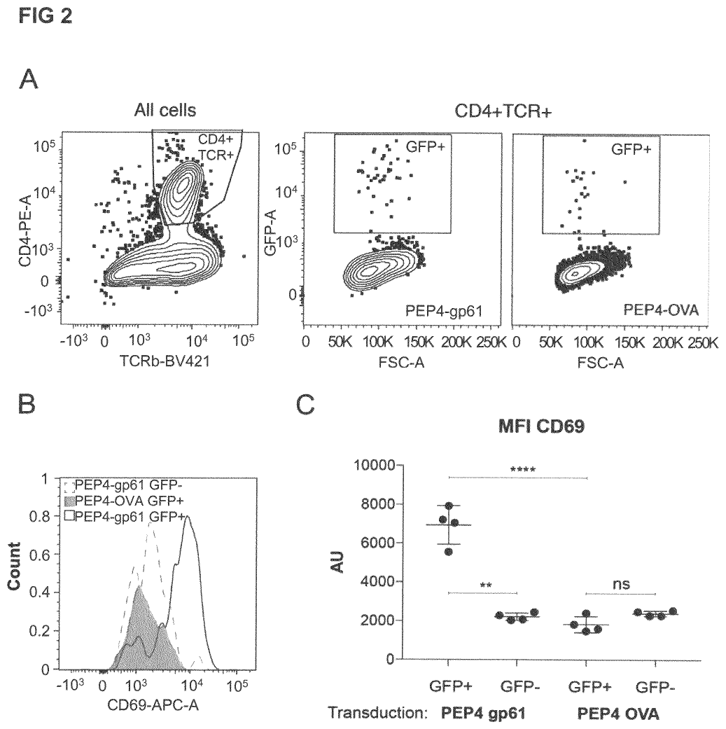

In particular, CD4- or CD8-negative T cell hybridomas may be used, particularly CD4- or CD8-negative T-cell hybridomas carrying a fluorescent reporter. However, this is not a prerequisite as CD4+ cells can be used as well (FIG. 2). A suitable fluorescent reporter for T cell activation is, for example, the nur77 fluorescent reporter, NFAT fluorescent reporter, or any other suitable reporter molecule (e.g. CD69).

In a second step, the polyclonal T cells expressing the library of polynucleotides of the invention are cultured in the presence of an antigen presenting cell that comprises a major histocompatibility complex on its cell surface. As mentioned above, the MHC molecule on the surface of the APC is required for the formation of a peptide-MHC complex with the peptide that is comprised in the molecule of the invention, such that the peptide-MHC complex can stimulate the T cell receptor on the cell surface of the T cell.

The term “antigen presenting cell”, as used herein, broadly refers to any cell that comprises a major histocompatibility complex on its cell surface. Thus, the antigen presenting cell may be a T cell itself. In certain embodiments, the antigen presenting cell is a bone marrow-derived primary dendritic cell (BMDC). It is preferred, that the MHC molecule comprised on the surface of the APC is either an MHC class I or an MHC class II molecule. In case the MHC molecule is an MHC class I molecule, it is preferred that the T cells express a polynucleotide encoding a chimeric molecule comprising the co-receptor CD8. In case the MHC molecule is an MHC class II molecule, it is preferred that the T cells express a polynucleotide encoding a chimeric molecule comprising the co-receptors CD4 or LAG3.

The term “contacting,” as used herein, refers to the act of bringing together two or more components, such as the T cells and the APCs, by dissolving, mixing, suspending, blending, slurrying, or stirring. A T cell expressing a polynucleotide of the invention is said to be contacted with an APC, if the two cells are in close enough proximity such that the peptide attached to the co-receptor of the T cell can form a peptide-MHC complex with the MHC molecule comprised on the surface of the APC. Preferably, cells are contacted in a solution, such as a cell culturing medium. The cells may be contacted for any amount of time that is sufficient for a T cell to be activated. In certain embodiments, the T cells are contacted with APCs for several hours or days such that the T cells in the culture that get activated upon contacting with an APC, proliferate and can be enriched. Thus, in certain embodiments, the term “contacting” is used interchangeably with the term “co-culturing”. That is, contacting a population of T cells synthesizing the chimeric molecule of the invention with an APC may be achieved by co-cultured the cells in liquid medium for a defined amount of time. Preferably, co-culturing leads to activation of T cells expressing a cognate TCR-peptide pair and, thus, results in proliferation and enrichment of these T cells.

In a fourth step, the T cells that have been activated upon contacting with an APC are isolated. As shown in Example 1, only the T cells that synthesize a chimeric molecule comprising an antigenic peptide that is recognized by their T cell receptor get activated in the presence of an APC. Thus, the contacting with an APC only leads to the activation and proliferation of T cells that express a cognate antigenic peptide. These T cells can then be isolated from the T cells that have not been activated in the presence of the APC to determine the cognate TCR-peptide pairs of the isolated T cells. The skilled person is aware of methods to isolate activated and enriched T cells.

For example, activated and enriched T cells can be identified by flow cytometry such as FACS sorting via the expression of activation markers, such as CD69, CD44 or CD25 and/or reporter proteins such as GFP, mCherry, mTomato, dsRed, or other suitable activation markers or reporter proteins, driven by, for example, promoters comprising NFAT or Nur77 binding sequences.

In particular, activation can be measured by fluorescence activated cell sorting (FAGS).

FACS refers to a method of separating a population of cells into one or more sub-populations based on the presence, absence, or level of one or more specific polypeptides expressed by the cells. FACS relies on optical properties, including fluorescence, of individual cells in order to sort the cells into sub-populations. Cell sorters suitable for carrying out a method described herein are well-known in the art and commercially available. Exemplary cell sorters include MoFlo sorter (DakoCytomation, Eon Collins, Colo.), FACSAria™, FACSArray™, FACS Vantage™, BD™ LSR II, and FACSCaiibur™ (BD Biosciences, San Jose, Calif.) and other equivalent cell sorters produced by other commercial vendors such as Sony, Bio-Rad, and Beckman Coulter.

Alternatively, T-cells comprising peptides efficiently presented by the MHC may be enriched by MACS-based cell sorting.

“MACS” refers to a method of separating a population of cells into one or more sub-populations based on the presence, absence, or level of one or more MACS-selectable polypeptides expressed by the cells. MACS relies on magnetic susceptibility properties of tagged individual cells in order to sort the cells into sub-populations. For MACS, magnetic beads (such as those available from Miltenyi Biotec Bergisch Gladbach, Germany; 130-048-402) can be used as labels. MACS cell sorters suitable for carrying out a method described herein are well-known in the art and commercially available. Exemplary MACS cell sorters include autoMACS Pro Separator (Miltenyi Biotec).

The sorting results in a population of non-fluorescent cells and at least one population of fluorescent cells, depending on how many fluorescent labels were used. The presence of at least one cell population with fluorescent cells is indicative that at least one candidate peptide is efficiently presented by APCs. Thus, FACS enables sorting of the population of cells to produce a population of cells enriched in T-cells comprising peptides efficiently presented by the MHC.

In a fourth step, the DNA of isolated T cells is sequenced. As described above, the T cells that are activated and enriched in the presence of an APC comprise a cognate TCR-peptide pair. Thus, sequencing the activated T cells that have been isolated in the previous step provides information about which peptide activates which T cell receptor.

The sequence of the TCRs and the corresponding cognate peptides can be obtained by single cell RNA/DNA sequencing of such a population of enriched T-cells.

Methods of DNA isolation and sequencing are known to those skilled in the art.

In general, the aim is to separate DNA present in the nucleus of the cell from other cellular components. The isolation of DNA usually begins with lysis or breakdown of cells. This process is essential for the destruction of protein structures and allows for release of nucleic acids from the nucleus. Lysis is carried out in a salt solution, containing detergents to denature proteins or proteases (enzymes digesting proteins), such as Proteinase K, or in some cases both. It results in the breakdown of cells and dissolving of membranes. Methods of DNA isolation include, but are not limited to, phenol:chloroform extraction, high salt precipitation, alkaline denaturation, ion exchange column chromatography, resin binding, and paramagnetic bead binding.

Methods of cDNA generation are known to those skilled in the art. In general, the aim is to convert the isolated RNA present in the cells to DNA, so called copy-DNA, in order to use it as template for polymerase chain reaction (PCR), The isolation of RNA usually begins with lysis or breakdown of cells. This process is essential for the destruction of protein structures and allows for release of nucleic acids from it. Lysis is usually carried out in Phenol containing solution (e.g. TRIzol™). It results in the breakdown of cells and dissolving of membranes and allows the separation of RNA from other cellular components. The isolated RNA is then converted into cDNA by reverse transcriptase (e.g. Superscript™, Goscript™)

The sequence of the candidate peptides carried by the activated T cells (which bind to the MHC complexes presented on the antigen presenting cell surface) is then amplified by PCR and may be sequenced by any method known in the art.

The sequence of the candidate peptides may be determined by digital PCR. Digital polymerase chain reaction (digital PCR, DigitalPCR, dPCR, or dePCR) is a refinement of conventional polymerase chain reaction methods that can be used to directly quantify and clonally amplify nucleic acids including DNA, cDNA or RNA.

Sequencing may also be performed using microfluidics. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets.

Sequencing may also be performed using Second Generation Sequencing (or Next Generation or Next-Gen), Third Generation (or Next-Next-Gen), or Fourth Generation (or N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology. Morozova and Marra provide a review of some such technologies in Genomics, 92: 255 (2008).

Prior to, following or concurrently with sequencing, nucleic acids may be amplified. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reverse transcribed to DNA prior to amplification (e.g., RT-PCR), whereas other amplification techniques directly amplify RNA (e.g., TMA and NASBA).

In a final step, the cognate TCR-peptide pairs of the activated and enriched T cells are identified by analyzing the sequencing data. The skilled person is aware of bioinformatic tools to analyze sequencing data. Thus, with the above described method, it will be possible to identify multiple cognate TCR-peptide pair in a single experiment.

In certain embodiments, the method according to the invention may be used for the identification of previously unknown antigenic peptides. For that, a polynucleotide library encoding a co-receptor protein attached to a peptide, wherein at least two polypeptides of the library encode peptides with different amino acid sequences, may be introduced into a population of polyclonal T cells.

In certain embodiments, the peptides in the library may be encoded by random DNA or cDNA molecules. The random DNA or cDNA molecules may, for example, be obtained from a cell, in particular an antigen presenting cell such as a tumor cell or a cell that has been infected with a pathogen, as described above. Further, the random DNA molecules may be DNA molecules or fragments of DNA molecules encoding a peptide or protein for immunogenicity testing.

In other embodiments, the peptide-encoding polynucleotides of the library may be obtained by chemical synthesis. For example, the sequences of the polynucleotides encoding the peptides of the library may be designed computationally based on exome data as described above. Alternatively, the sequences of the polynucleotides encoding the peptides of the library may be designed based on the sequence of peptides that have been isolated from an antigen presenting cell as described above.

The library of polynucleotides may be introduced into any population of T cells. In particular, the population of T cells may be a population of T cells that has been obtained from the same subject as the DNA or cDNA molecules encoding the peptides of the library.

In certain embodiments, the methods of the invention may be used for the identification of previously unknown tumor antigens. In this case, it is preferred that the sequence information of the peptides that are encoded in the library of polynucleotides is obtained from a tumor cell. To identify T cell receptors that specifically bind to these peptides, it is further preferred that the library of polynucleotides is screened against monoclonal or polyclonal T cells that have been obtained from a subject, in particular a subject suffering from cancer, in particular the same subject the tumor cell that was used for the generation of the library of polynucleotides has been obtained from.

In certain embodiments, the methods of the invention may be used for the identification of previously unknown pathogen-associated antigens. In this case, it is preferred that the sequence information of the peptides that are encoded in the library of polynucleotides is obtained from a cell that has been infected with a pathogen. To identify T cell receptors that specifically bind to these peptides, it is further preferred that the library of polynucleotides is screened against monoclonal or polyclonal T cells that have been obtained from a subject, in particular a subject suffering from an infectious disease. Preferably, the cell that is used for the generation of the library and the population of monoclonal or polyclonal T cells have been obtained from subjects suffering from the same infectious disease, more preferably from the same subject.

In certain embodiments, the methods of the invention may be used for the identification of antigenic peptides that are involved in the onset and progression of autoimmune diseases. Autoimmune diseases may be caused by pathogenic T cells that get activated by antigens that are present on healthy cell. Thus, identifying antigenic peptides that are frequently recognized by pathogenic T cells may facilitate the development of agents that block this recognition, such as binding agents that bind to the antigenic peptides and prevent the recognition by a pathogenic T cell. Accordingly, the method of the invention may be used to identify antigenic peptides that can be recognized by pathogenic T cells. For that, a polynucleotide library may be screened against a monoclonal or polyclonal population of pathogenic T cells. It is preferred that the monoclonal or polyclonal population of pathogenic T cells is obtained from a patient suffering from an autoimmune disease. Alternatively, polynucleotides encoding one or more known pathogenic T cell receptors may be introduced into non-pathogenic T cells to obtain a population of pathogenic T cells. The term “pathogenic T cell”, as used herein, refers to a T cell that responds to a self-antigen during the onset or progression of an autoimmune disease.

In a particular embodiment, the invention relates to a method for identifying at least one antigen-specific T cell receptor, the method comprising the steps of: (a) providing polyclonal T-cells of interest expressing a polynucleotide of the invention; (b) contacting the T-cells of step a) with antigen presenting cells (APC) comprising a major histocompatibility complex (MHC); (c) isolating at least one T-cell that is activated upon contacting with the APCs in step (b); (d) sequencing of the TCR loci of the at least one T cell isolated in step (c); and (e) identifying at least one T cell receptor encoded by the TCR loci of the at least one T cell to be antigen-specific.

Instead of screening various T cell receptors against multiple antigenic peptides simultaneously, the chimeric molecules of the invention may also be used for the identification of T cell receptors that are stimulated by a specific antigen. For that, a polynucleotide encoding a co-receptor protein attached to a specific peptide is expressed in a population of polyclonal T cells.

Accordingly, the method of the invention may be used for the identification of T cell receptors with high specificity for a known antigenic target. For example, the known antigenic target may be an antigenic peptide that has been determined to be present on the surface of an antigen presenting cell of a subject. In particular, the antigenic peptide may be a pathogen-derived peptide that is presented by an MHC molecule on the surface of a cell that has been infected with the pathogen. Alternatively, the antigenic peptide may be a peptide that is specifically presented by an MHC molecule on the surface of a tumor cell, but not on the surface of a healthy cell. By attaching a peptide to a co-receptor that comprises an amino acid sequence that is identical to the amino acid sequence of the antigenic peptides that are presented on the infected cell or the tumor cell, it is possible to identify T cell receptors with a high specificity for these antigenic peptides.

The antigen-specific T cell receptors that may be identified with the method of the invention may be naturally occurring T cell receptors. For example, in certain embodiments, the population of polyclonal T cells may be isolated from a subject. In certain embodiments, the population of polyclonal T cells may be isolated from a subject that has been determined to comprise infected cells or tumor cells that display the antigenic peptides on their cell surfaces.

Alternatively, the population of polyclonal T cells may be obtained by genetic engineering. For example, the population of polyclonal T cells may be obtained by introducing a library of T cell receptors into a population of T cells, in particular TCR negative T cells. In certain embodiments, the library of T cell receptors may be generated based on the sequence of a T cell receptor that already shows a certain degree of binding to the antigenic peptide that is presented by an MHC molecule on the surface of an APC. That is, the library may be generated by introducing random or targeted mutations into the DNA sequence encoding the T cell receptor, with the aim to identify a mutated variant of the T cell receptor that is stimulated by the antigenic peptide more efficiently.

In a particular embodiment, the invention relates to a method for identifying at least one T cell-specific antigen, the method comprising the steps of: (a) providing monoclonal T-cells of interest expressing a polynucleotide of the invention or a library of polynucleotides of the invention; (b) contacting the T cells of step (a) with antigen presenting cells (APC) comprising a major histocompatibility complex (MHC); (c) isolating at least one T cell that is activated upon contacting with the APCs in step (b); (d) sequencing the part of the polynucleotide encoding the peptide attached to the N-terminus of a CD4, LAG-3 or CD8 co-receptor protein of the at least one T cell isolated in step (c); and (e) identifying at least one peptide encoded by the polynucleotide comprised in the at least one T cell to be a T cell-specific antigen.

That is, in certain embodiments, the chimeric molecule of the invention is used to identify peptides that efficiently stimulate a specific T cell receptor. For that, a population of monoclonal T cells may be screened against the library of polynucleotides of the invention. A population of monoclonal T cells is a group of more than one T cell, wherein essentially all T cells in the population synthesize an identical T cell receptor. Accordingly, the method of the invention may be used to identify peptides from a library of peptides that efficiently stimulate a specific T cell receptor. For example, the library of peptides may be encoded by the library of polynucleotides of the invention, wherein at least two polynucleotides of the library encode an identical co-receptor protein attached to a peptide with a different amino acid sequence.

In certain embodiments, the peptides that are attached to the co-receptor protein may differ in their amino acid sequence in that they comprise at least two different antigenic peptides that are known to be presented by an MHC molecule. In particular, the antigenic peptides may be peptides that are derived from a tumor cell or from a cell that has been infected with a pathogen.

In other embodiments, the peptides that are attached to the co-receptor protein may differ in their amino acid sequence in that they are mutated variants of a known antigenic peptide. That is, the method of the invention may be used to identify mutated variants of known antigenic peptide that result in improved stimulation of the cognate T cell receptor. For example random or targeted mutations may be introduced into a peptide that comprises an amino sequence of a known antigenic peptide to identify peptides that result in more efficient stimulation of a known T cell receptor.

In a particular embodiment, the invention relates to the method of the invention, wherein the APC is an autologous or a heterologous APC.

That is, the APC that is contacted with the T cells in the methods of the invention may be an autologous or a heterologous APC. An “autologous APC”, as used herein, is an APC that has been obtained from the same subject as the T cells that the APC is contacted with. A “heterologous APC”, as used herein, is an APC that has been obtained from a different subject as the T cells that the APC is contacted with.

In a particular embodiment, the invention relates to the method of the invention, wherein the APC is a genetically modified autologous or heterologous cell or cell line, expressing a mutated MHC molecule.

The autologous or heterologous APC that is contacted with the T cells in the methods of the invention may be a genetically modified APC that expresses a mutated MHC molecule.

In a particular embodiment, the invention relates to the method of the invention, wherein the mutated MHC molecule is a MHC class II molecule comprising the extracellular MHC class II alpha chain and a native or heterologous transmembrane domain, as well as the extracellular MHC class II beta chain and a native or heterologous transmembrane domain.

That is, the mutated MHC class II molecule may comprise an MHC class II alpha chain and the extracellular domain of an MHC class II beta chain fused to a native or heterologous transmembrane domain. Alternatively, the mutated MHC class II molecule may comprise an MHC class II beta chain and the extracellular domain of an MHC class II alpha chain fused to a native or heterologous transmembrane domain. Alternatively, the mutated MHC class II molecule may comprise the extracellular domain of an MHC class II alpha chain fused to a native or heterologous transmembrane domain and the extracellular domain of the MHC class II beta chain fused to a native or heterologous transmembrane domain.

In a particular embodiment, the invention relates to the method of the invention, wherein the mutated MHC molecule is a MHC class I molecule comprising the extracellular MHC class I alpha chain and a native or heterologous transmembrane domain, as well as beta-2 microglobulin.

That is, an extracellular domain of an MHC class I or II molecule is said to be fused to a native transmembrane domain, if the extracellular domain of the MHC class I or class II molecule is covalently linked to a transmembrane domain of a different MHC class I or class II molecule, respectively. An extracellular domain of an MHC class I or II molecule is said to be fused to a heterologous transmembrane domain, if the extracellular domain of the MHC class I or II molecule is covalently linked to a transmembrane domain from any other transmembrane protein, in particular any other mammalian transmembrane protein. The skilled person is aware of methods to fuse the extracellular domain of a first protein to the transmembrane domain of a second protein.

In a particular embodiment, the invention relates to the method of the invention, wherein the co-receptor protein encoded by the polynucleotide or the library of polynucleotides is CD8, and wherein the MHC molecule comprised in the APC is a MHC class I molecule.

That is, the co-receptor protein CD8 is known to facilitate the binding between peptide-MHC class I complexes and a cognate TCR. Thus, it is preferred within the present invention that the polynucleotide or the library of polynucleotides that is introduced into the T cells encodes the co-receptor protein CD8 if the resulting cells are to be contacted with an APC that comprises an MHC class I molecule.

MHC Class I molecules are expressed on the surface of cells in all jawed vertebrates, and are responsible for displaying antigens to T cells. The genes encoding the MHC molecules are found in the MHC region of the vertebrate genome, although the gene composition and genomic arrangement vary widely.

In humans, these genes are referred to as human leukocyte antigen (HLA) genes. The most intensely studied HLA genes are the nine so-called classical MHC genes: HLA-A, HLA-B, HLA-C, HLA-DPA 1, HLA-DPB 1, HLA-DQA 1, HLA-DQB 1, HLA-DRA, and HLA-DRB 1. In humans, the MHC is divided into three regions: Class I, II, and III. The A, B, and C genes belong to MHC class I, whereas the six D genes belong to class II.

MHC Class I protein molecules are heterodimers comprising two polypeptide chains: a highly polymorphic a chain (comprising 3 domains: at a2 and a3) and a non-covalently associated P2-microglobulin. Human MHC Class I protein molecules may be referred to in the art as “HLA molecules”, or “HLA protein molecules”.

Accordingly, the term “MHC Class I molecule” as used herein includes all mammalian MHC Class I molecules including human and non-human. Preferably the MHC Class I molecule is a human MHC Class I molecule (HLA protein molecule).

MHC Class I molecules are responsible for binding and presenting antigens on the cell surface and therefore exist with or without bound antigen. Accordingly, the term MHC Class I molecule refers the MHC Class I molecule either on its own, or when bound to an antigen.

The MHC Class I molecule may be an HLA molecule. Preferably the HLA molecule is a product of the HLA-A gene. The HLA-A gene is polyallelic and as such, a variety of differences in the a chain of the encoded protein exist within the population.

Preferably the HLA molecule bound by the first portion according to the invention is a product of the HLA-A2 gene. HLA-A2 is a HLA serotype within the HLA-A ‘A’ serotype group and is encoded by the H LA-A 02 allele group including the HLA-A0201, HLA-A0202, HLA-A0203, HLA-A0205, HLA-A0206, HLA-A0207 and HLA-A021 1 gene products. HLA-A2 is very common in the Caucasian population (40-50%) and provides an ideal cellular target for the first portion because it will be suitable for use in many combinations of donor and recipient. This approach would be suitable to roughly a quarter of all transplantation cases in the Caucasian population.

Accordingly, any member of the HLA-A2 allele group is encompassed by the term ‘HLA-A2’.

In a particular embodiment, the invention relates to the method of the invention, wherein the co-receptor protein encoded by the polynucleotide is CD4 or LAG-3, and wherein the MHC molecule expressed by the APC is a MHC class II molecule.

It is further preferred that the polynucleotide or the library of polynucleotides that is introduced into the T cells encode the co-receptor proteins CD4 or LAGS if the resulting cells are to be contacted with an APC that comprises an MHC class II molecule.

The MHC class II molecule is composed of two membrane spanning polypeptide chains, α and β, of similar size (about 30000 Da). Each chain consists of two domains, where α1 and β1 forms a 9-pocket peptide-binding cleft, where pocket 1, 4, 6 and 9 are considered as major peptide binding pockets. The α2 and β2, like the α2 and β2m in the MHC class I molecules, have amino acid sequence and structural similarities to immunoglobulin constant domains. In contrast to MHC class I complexes, where the ends of the antigenic peptide is buried, peptide-ends in MHC class II complexes are not. HLA-DR, DQ and DP are the human class II molecules, H-2A, M and E are those of the mice.

In a particular embodiment, the invention relates to a method for treating a subject suffering from cancer or an infectious disease, the method comprising the steps of: (a) Identifying at least one antigen-specific T cell receptor and/or at least one T cell-specific antigen with the methods of the invention; (b) administering to the subject suffering from cancer or from an infectious disease the at least one T cell receptor and/or T cell-specific antigen identified in step (a).

That is, the method of the invention may be used to identify antigenic peptides or antigen-specific T cell receptors that can be used in the treatment of a subject suffering from cancer or from an infectious disease. For example, the methods of the invention may be used to identify naturally occurring or engineered T cell receptors that are efficiently stimulated by an antigenic peptide that is presented by MHC molecules on the subject's tumor cells or on cells that have been infected with a pathogen. In this case, the identified antigen-specific T cell receptor may be administered to the subject suffering from cancer to attack the tumor cells in that subject or to the subject suffering from an infectious disease to attack cells that have been infected with the pathogen.

Alternatively, the methods of the invention may be used to identify previously unknown antigenic peptides. In particular, the methods of the invention may be used to identify previously unknown antigenic peptides that are expressed by an APC, in particular by a tumor cell or by a cell that has been infected with a pathogen.

The methods of the invention may further be used to determine if a newly identified antigenic peptide is recognized by a T cell, in particular a T cell that has been obtained from the same subject as the tumor cell or the infected cell. If a previously unknown cognate TCR-peptide pair is identified in a subject suffering from cancer or an infectious disease by using the methods of the invention, said subject may be treated with the antigenic peptide or its cognate T cell receptor.

In certain embodiments, it is preferred that the monoclonal or polyclonal T cells that have been used for the identification of the antigenic peptide and/or the antigen specific T cell receptor have been obtained from the patient suffering from cancer or from an infectious disease. In further embodiments, it is preferred that the library of polynucleotides that is introduced into the T cells comprises polynucleotide sequences that have been obtained from APCs of the subject suffering from cancer or from an infectious disease, in particular from tumor cells of the subject suffering from cancer or from cells that have been infected with the pathogen.

Cancers that may be treated with the method of the invention include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the lymphocytes of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases).

Within the present invention, the infectious disease may be caused by any pathogen. In certain embodiments, the infectious disease is caused by a viral pathogen. In certain embodiments, the viral pathogen cytomegalovirus (CMV), Epstein-Barr virus (EBV), adenoviruses, polyomaviruses, Varizella-Zoster virus (VZV), human herpesvirus (HHV), human immunodeficiency virus (HIV) or influenza virus.

In a particular embodiment, the invention relates to the method of the invention, wherein the antigen-specific T cell receptor is administered to the subject by virus-mediated gene delivery.

A naturally occurring or engineered T cell receptor that has been identified with the methods of the invention to efficiently bind an antigen that is presented on the surface of a tumor cell that has been obtained from a subject suffering from cancer or on the surface of a pathogen-infected cell that has been obtained from a subject may be administered to that subject by any route. For example, the T cell receptor may be administered to the subject by virus-mediated gene delivery. For that, it is preferred that a population of T cells is first isolated from the subject. The genes encoding the antigen-specific TCR are introduced into the population of T cells by virus-mediated gene delivery and the T cells that received the genes and synthesize the antigen-specific T cell receptor are then re-introduced into the subject. The skilled person is aware of methods and suitable viral vectors for virus-mediated gene delivery. In particular, lentiviral or adeno-associated virus-based vectors may be used for the delivery of TCR genes to a T cell.

Alternatively, the antigen-specific T cell receptor may be a soluble antigen-specific T cell receptor that is directly administered to the subject. In a particular embodiment, the soluble antigen-specific T cell receptor is conjugated to another molecule, such as a pharmaceutical compound.

In a particular embodiment, the invention relates to the method of the invention, wherein the T cell-specific antigen is administered to the subject in form of a peptide or in form of a polynucleotide encoding a peptide.

An antigenic peptide that has been identified with the methods of the invention may be administered to a subject suffering from cancer or an infectious disease in any way. That is, the antigenic peptide may be administered to the subject in form of a peptide, such that the peptide is taken up and presented by an antigen presenting cell which, in turn, may result in the activation of antigen-specific T cells. Alternatively, the antigenic peptide may also be administered to the subject in form of a larger peptide or protein comprising the antigenic peptide. The larger peptide or protein may then be taken up by an antigen presenting cell and processed, such that the antigenic peptide is presented by an MHC on the surface of the antigen presenting cell. Alternatively, the antigenic peptide may be administered to the subject in form of a polynucleotide, which may be taken up by an APC such that the antigenic peptide is expressed and presented by the APC. In certain embodiments, the polynucleotide encoding the antigen-specific peptide is a DNA molecule. In other embodiments, the polynucleotide encoding the antigen-specific peptide is an RNA molecule, in particular an mRNA molecule. In certain embodiments, the polynucleotide comprises further regulatory elements or coding sequences.

In a particular embodiment, the invention relates to the method of the invention, wherein the peptide or the polynucleotide encoding the peptide is attached to a compound that improves delivery of the peptide or polynucleotide encoding the peptide to an APC.

The peptide or the polynucleotide encoding the peptide may be attached to any compound that facilitates the delivery of the peptide or the polynucleotide to the APC. That is, the peptide or polynucleotide may be attached, for example, to a targeting moiety that directs the peptide or polynucleotide to the APC.

The term “administration,” as used herein to refer to the delivery of an inventive TCR material or antigenic peptide to a subject, is not limited to any particular route but rather refers to any route accepted as appropriate by the medical community. The term “subject” as used herein denotes any animal, preferably a mammal, and more preferably a human. Examples of subjects include humans, non-human primates, rodents, guinea pigs, rabbits, sheep, pigs, goats, cows, horses, dogs and cats. Within the present invention, the term “subject” is used interchangeably with the term “patient”.

The term “treating” as used herein refers to any improvement of a disease or disorder, such as cancer or an infectious disease, that occurs in a treated subject compared to an untreated subject. Such an improvement can be a prevention of a worsening or progression of the said disease or disorder. Moreover, such an improvement may also be an amelioration or cure of the disease or disorder or its accompanying symptoms. It will be understood that a treatment may not be successful for 100% of the subjects to be treated. The term, however, requires that the treatment is successful for a statistically significant portion of the subjects (e.g. a cohort in a clinical study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%© or at least 99%©. The p-values are, preferably, 0.05, 0.01, 0.005, or 0.0001.
B1. A chimeric molecule comprising the CD4, LAG3 or CD8 co-receptor protein and a peptide attached to the N-terminus of the co-receptor.
B2. The chimeric molecule of embodiment B1, wherein the peptide is attached to the N-terminus of the co-receptor via a linker.
B3. The chimeric molecule of embodiment B1 or embodiment B2 wherein the peptide is encoded by a given cDNA or DNA molecule,
B4. The chimeric molecule of embodiment B3, wherein the cDNA or DNA is derived from fragmented cDNA or DNA molecule.
B5. The chimeric molecule of any one of embodiments B1 to B4, wherein the peptide is a random peptide.
B6. The chimeric molecule of any one of embodiments B1 to B5, wherein the peptide is derived from a peptide library.
B7. The chimeric molecule of any one of embodiments B1 to B6, wherein the peptide has between 6 to 200 amino acid residues.
B8. The chimeric molecule of any one of embodiments B1-B7, wherein the peptide is encoded by a DNA comprising a SNP present in a tumor DNA.
B9. A chimeric DNA construct comprising a DNA encoding the chimeric molecule of any one of embodiments B1-B8.
B10. A library of chimeric molecules comprising one or more of the molecules defined in any one of embodiments B1-B8.
B11. A method for simultaneous detection and enrichment of antigen-specific T cells and the peptides specifically recognized by their T cell receptors (TCRs), which comprises
   a) providing polyclonal T-cells of interest overexpressing a chimeric DNA construct encoding the chimeric molecule of any one of embodiments B1-B8.
   b) culturing the T-cells overexpressing the chimeric DNA construct of step a) in the presence of antigen presenting cells (APC) expressing the major histocompatibility complex (MHC) comprising a peptide-binding groove such that a complex is formed between the peptide attached to the N-terminus of a CD4, LAG-3 or CD8 co-receptor protein and the peptide-binding groove of MHC, which upon recognition by the TCR, leads to activation of the T-cells;
   c) isolating the T-cells activated and expanded in the co-culture step b); and optionally
   d) sequencing the DNA of the isolated T cells to obtain information about the TCR sequences and peptide sequences attached to the CD4, LAG-3 or CD8 co-receptors present in these T cells.
B12. The method of embodiment B11, wherein the activated T-cell comprises a TCR, which recognizes the MHC peptide complex formed in step b).
B13. A method for identification of antigen-specific TCR sequences which comprises
   a) providing polyclonal T-cells of interest overexpressing a chimeric DNA construct encoding the chimeric molecule of any one of embodiments B1-B8;
   b) culturing the T-cells overexpressing the chimeric DNA construct of step a) in the presence of an antigen presenting cells (APC) expressing the major histocompatibility complex (MHC) comprising a peptide-binding groove such that a complex is formed between the peptide attached to the N-terminus of a CD4, LAG-3 or CD8 co-receptor protein and the peptide-binding groove of MHC, which upon recognition by the TCR, leads to activation of the T-cells;
   c) identifying and sorting of T cells activated and expanded in the co-culture step b) via expression of activation markers or reporter proteins;
   d) sequencing of the TCR loci of the T cells identified and sorted in step c).
B14. A method for the identification of T cell-specific antigens which comprises
   a) providing monoclonal T-cells of interest overexpressing a chimeric DNA construct encoding the chimeric molecule of any one of embodiments B1-B8;
   b) culturing the T-cells overexpressing the chimeric DNA construct of step a) in the presence of an antigen presenting cells (APC) expressing the major histocompatibility complex (MHC) comprising a peptide-binding groove such that a complex is formed between the peptide attached to the N-terminus of a CD4, LAG-3 or CD8 co-receptor protein and the peptide-binding groove of MHC, which upon recognition by the TCR, leads to activation of the T-cells;
   c) identifying and sorting of T cells activated in the co-culture of step b) via the expression of activation markers or reporter proteins;
   d) isolating DNA/RNA present in the T cells identified and sorted in step c) e) amplifying (by PCR or rtPCR) the fragment encoding the chimeric co-receptor; and
   f) sequencing of the part encoding the peptide attached to the N-terminus of a CD4, LAG-3 or CD8 co-receptor protein.
B15. The method of any one of embodiments B11 to B14, wherein the APC is an autologous APC.
B16. The method of any one of embodiments B11 to B14, wherein the APC is a heterologous APC.
B17. The method of any one of embodiments B11 to B14, wherein the APC is a genetically modified autologous or heterologous cell or cell line, overexpressing a mutated MHC molecule.
B18. The method of any one of embodiments B11 to B17, wherein in step a) the chimeric DNA constructs, overexpressed in T-cells of interest, encode a peptide library.
B19. The method of embodiment B18, wherein the peptide library is a library as defined in embodiment B10.
B20, The method of any one of embodiments B11-B19, wherein the co-receptor is CD8 and the MHC molecule is a MHC class I molecule.
B21. The method of any one of embodiments B11-B19, wherein the co-receptor is CD4, or LAG-3, and the MHC molecule is a MHC class II molecule.
B22. The method of any one of embodiments B17 to B21, wherein the mutated MHC molecule is a MHC class II molecule comprising the extracellular MHC class II alpha chain and a native or heterologous transmembrane domain, as well as the extracellular MHC class II beta chain and a native or heterologous transmembrane domain.
B23. The method of any one of embodiments B17 to B21, wherein the mutated MHC molecule is a MHC class I molecule, comprising the extracellular MHC class I alpha chain and a native or heterologous transmembrane domain, as well as beta-2 microglobulin.

The present invention provides methods for simultaneously detecting and enriching antigen-specific T cells and the peptides specifically recognized by their T cell receptors (TCRs), and for the identification of T cell-specific antigens for in vivo and/or in vitro interventions including vaccination, induction of immunological tolerance, blocking of TCRs and MHC-mediated toxin delivery, for immunogenicity testing and other in vitro T-cell reactivity tests. The present invention also relates to the chimeric molecules used in said methods.

In particular, the method comprises the steps of
a) providing polyclonal or monoclonal T-cells of interest overexpressing a chimeric DNA construct comprising the chimeric molecule of any one of embodiments B1-B4;
b) culturing the T-cells overexpressing the chimeric DNA construct of step a) in the presence of an antigen presenting cell (APC) expressing the major histocompatibility complex (MHC) comprising a peptide-binding groove such that a complex is formed between the peptide attached to the N-terminus of a CD4, LAG-3 or CD8 co-receptor protein and the peptide-binding groove of MHC, which upon recognition by the TCR, leads to activation of the T-cells;
c) isolating T cells activated in the co-culture of step b), particularly by flow cytometry and FACS sorting the T cells via the expression of activation markers, such as CD69, CD44 or CD25 or reporter proteins such as GFP, mCherry, mTomato, dsRed, etc.; and
d) isolating DNA/RNA present in the T cells identified and sorted in step c)
e) amplifying (by PCR or rtPCR) the fragment encoding the chimeric co-receptor and
f) sequencing of the part encoding the peptide attached to the N-terminus of a CD4, LAG-3 or CD8 co-receptor protein.

In various specific embodiments of the invention, the T cells activated in the co-culture of step b) may be isolated by flow cytometry and FACS sorting of the T cells via the expression of activation markers such as, for example, CD69, CD44 or CD25 and/or reporter proteins such as, for example, GFP, mCherry, mTomato, dsRed, or other suitable activation markers or reporter proteins.

The art known methods are severely limited as it comes to parallel processing as they do not allow simultaneous screening of many TCRs against many epitopes. The TCRs of interest have to be screened against epitope libraries one by one. Vice-versa, finding TCRs reactive to epitopes of interest, requires the epitopes to be screened against TCR libraries one by one.

The present invention provides for the first time a method which allows simultaneous detection and enrichment of antigen-specific T cells and identification of the TCRs and their cognate epitopes.

This is achieved by expressing chimeric molecules comprising the CD4, LAG-3 or CD8 co-receptor proteins and a library of peptides attached to the N-terminus of these co-receptors in T cells of interest. Also TCR-negative T cell lines overexpressing libraries of TCRs may be used.

In particular, a T cell of interest may be a T cell isolated fro blood, spleen, lymph nodes or tumor tissue or from any other suitable source.

In particular, the above described chimeric molecules are comprised in a recombinant expression vector.

The CD4, LAG-3 or CD8 co-receptor proteins re in particular full length CD4, LAG-3 or CD8 co-receptor proteins.

Tethering of the peptide to the N-terminus of the CD4, LAG-3 or CD8 co-receptors may be effected by use of a linker molecule.

In particular, the linker molecule comprises between about 5 to 30 amino acids. Suitable linker molecules are (G4S)₁, (G4S)₂, (G4S)₃, (G4S)₅, etc.

In particular, a GS-linker may be used ranging from 5 to 28 amino acids.

The peptide attached to the N-terminus of the co-receptor is in particular (i) a random peptide; (ii) a peptide derived from a given cDNA or DNA molecule; (iii) a peptide encoded by a fragmented cDNA or DNA molecule.

Fragmented cDNA or DNA molecules may be generated by random sheering or digestion of cDNA or DNA derived from tissue biopsies, cells or pathogens of interest.

For example, the peptide attached to the N-terminus of the co-receptor may be encoded by a DNA molecule or a fragmented DNA molecule comprising a SNP present in a tumor DNA.

Other peptides that may be used in the chimeric construct are, for example, without being limited to:
TSA obtained by exome sequencing used to identify TSAs that are uniquely present in a tumor;
a tumor-specific peptide carrying individual tumor-derived mutation(s). such as a single nucleotide variant (SNV). SNV including various mutations of p53, KRAS, and BRAF;
an antigen that causes an immune response, for example, the peptides can be derived from pathogens;
a compound undergoing immunogenicity testing;
a library of candidate peptides, wherein the library comprises mutant forms of native peptide(s).

In particular, a library of candidate peptides may be used in the methods disclosed herein, wherein the library comprises peptides generated by random sheering or digestion of cDNA or DNA derived from cells or pathogens of interest. Such a library would cover all peptides present in such cells.

The chimeric molecule comprising the CD4, LAG-3 or CD8 co-receptor protein and a peptide attached to the N-terminus of the co-receptor are expressed in T cells of interest.

In particular, recombinant expression vectors may be used, which are replicable DNA constructs comprising an assembly of (1) agent(s) having a regulatory role in gene expression, for example, promoters, operators, or enhancers, operatively linked to (2) a nucleotide sequence encoding a desired protein (such as the CD4, Lag-3 or CD8 with tethered peptides) which is transcribed into mRNA and translated into protein, and (3) appropriate transcription and translation initiation and termination sequences. The choice of promoter and other regulatory elements generally varies according to the intended reporter cell line. Expression vectors are often in the form of “plasmids” which refer to circular double stranded DNA loops which, in their vector form are not bound to the chromosome.

Eukaryote expression vectors, replicating episomally, such as pCEP4 or BKV, or other vectors derived from viruses, such as retroviruses e.g. pMY, pMX, pSIR, adenoviruses e.g. pAd, and the like, may be employed. In the expression vectors, regulatory elements controlling transcription or translation can be generally derived from mammalian, microbial, viral or insect genes. The ability to replicate, usually conferred by an origin of replication (e.g Epstein Barr virus latent origin of DNA replication), and a selection gene to facilitate recognition of transformants may additionally be incorporated. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters for use in eukaryotic host cells are known to those skilled in the art. Illustrative examples of such promoters include, but are not limited to, promoters from Simian Virus 40 (SV40), Mouse Mammary Tumor Virus (MMTV) promoter, Human Immunodeficiency Virus (HIV) promoters, such as the HIV Long Terminal Repeat (LTR) promoter, Moloney virus promoters, ALV promoters, cytomegalovirus (CMV) promoters, such as the CMV immediate early promoter, Epstein Barr Virus (EBV) promoter, Raus Sarcoma Virus (RSV) promoter, as well as promoters from human genes such as human actin, human myosin, human hemoglobin, human muscle creatine, and human metalothionein. Still other examples of suitable promoters include the CAG promoter (a hybrid promoter comprising a CMV enhancer, a chicken β-actin promoter, and a rabbit β-globin splicing acceptor, and poly(A) sequence).

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The chimeric DNA molecule comprising the CD4, LAG-3 or CD8 co-receptor protein and a peptide attached to the N-terminus of the co-receptor as described herein, particularly the chimeric DNA molecule comprised in an expression vector, may be introduced into T-cells of interest by transducing the chimeric DNA molecule, particularly the expression vector comprising the chimeric DNA molecule, into the host cell using standard techniques known in the art. Suitable methods are, for example, described in Sambrook et al., Molecular Cloning: A Laboratory Manual, Cold Spring Harbor, N.Y. (1989). To produce pseudo-retroviruses for transduction, packaging cell lines constantly expressing retroviral proteins GAG, POL and ENV (like for example the Phoenix cell line), are transiently transfected with constructs containing the viral genome composed of the LTRs, packaging signals and the genes of interest (in this case the peptide carrying MHC chains). Alternatively, suitable cell lines, like HEK, 3T3 or other, are transiently transfected with a mixture of vectors encoding separately the retroviral proteins GAG, POL and ENV and the viral genome composed of the LTRs, packaging signals and the genes of interest. These commonly used strategies ensure the production of defective pseudo-retroviruses which are able to infect target cells and introduce the genes of interest into their genomic DNA. However, the infected target cells are not able to produce retroviruses because the pseudo-retroviruses do not carry the gag, pol and env genes in their genome.

Alternatively, the chimeric DNA molecule, particularly the expression vector comprising the chimeric DNA molecule, can be introduced into T-cells of interest by transfection with reagents based on lipids, calcium phosphate, cationic polymers or DEAE-dextran, or by electroporation.

T cells that may be used in the herein disclosed method are for example, without being limited to, T cells isolated from blood, spleen, lymph nodes or tumor tissue.

In particular, CD4- or CD8-negative T cell hybridomas may be used, particularly CD4- or CD8-negative T-cell hybridomas carrying a fluorescent reporter. However, this is not a prerequisite as CD4+ cells can be used as well (FIG. 2).

A suitable fluorescent reporter for T cell activation is, for example, the nur77 fluorescent reporter, NFAT fluorescent reporter, or any other suitable reporter molecule.

Efficient expression of the fusion construct comprising the CD4, LAG-3 or CD8 co-receptor protein and the peptide attached to the N-terminus of the co-receptor protein on the cell surface may be determined by CD4-, LAG-3 or CD8-specific antibody staining. The antibody may be directly conjugated to a detectable label. Alternatively, a secondary antibody, conjugated to a detectable label and specific for the first antibody, may be contacted with the cells. Detectable labels suitable for use include any compound detectable by spectroscopic, photochemical, biochemical, immunochemical, electrical, optical or chemical means. Useful labels in the present invention include biotin, magnetic beads (e.g., Dynabeads™), fluorescent labels (e.g., fluorescein, texas red, rhodamine, green fluorescent protein, dansyl, umbelliferone, PE, APC, CY5, Cy7, PerCP, Alexa dyes and the like), enzymes (e.g., horse radish peroxidase, alkaline phosphatase and others), and colorimetric labels such as colloidal gold or colored glass or plastic (e.g., polystyrene, polypropylene, latex, etc.) beads. A variety of suitable fluorescent labels are further described in, for example, The Molecular Probes Handbook: A Guide to Fluorescent Probes and Labeling Technologies, 11th Edition.

When T-cells over-expressing the chimeric molecule as defined herein are cultured in the presence of autologous antigen presenting cells (APCs), peptides attached to the N-terminus of the CD4, LAG-3 or CD8 co-receptor protein are inserted into the groves of the MHC molecules expressed on the surface of the APCs such that they can be presented to the T-cells.

If the peptides are recognised by the TCR, the T-cells get activated and proliferate.

Accordingly, transfecting (by retroviral transduction, or electroporation) a construct comprising the chimeric molecule as described herein encoding a given peptide into polyclonal T cells leads exclusively to proliferation and enrichment of T cells comprising TCRs specific for that peptide.

The constructs used for transduction may encode not just a single peptide, but a library of peptides.

Transfecting (by retroviral transduction, or electroporation) constructs encoding a library of peptides into polyclonal T cells leads exclusively to proliferation and enrichment of T cells carrying peptides, which are specifically recognized by their TCRs.

As the stimulating peptides are attached to the N-terminus of the CD4, LAG-3 or CD8 co-receptor protein and are thus comprised within the T-cells, after sufficient time that only T cells carrying their cognate peptides will be left in culture.

Activated and enriched T cells can be identified by flow cytometry and FACS sorting via the expression of activation markers, such as CD69, CD44 or CD25 and/or reporter proteins such as GFP, mCherry, mTomato, dsRed, or other suitable activation markers of reporter proteins, driven by, for example, NFAT or Nur77 promoters;

In particular, activation can be measured by fluorescence activated cell sorting (FACS).

FACS refers to a method of separating a population of cells into one or more sub-populations based on the presence, absence, or level of one or more specific polypeptides expressed by the cells. FACS relies on optical properties, including fluorescence, of individual cells in order to sort the cells into sub-populations. Cell sorters suitable for carrying out a method described herein are well-known in the art and commercially available. Exemplary cell sorters include MoFlo sorter (DakoCytomation, Fori Collins, Colo.), FACSAria™, FACSArray™, FAGS Vantage™, BD™ LSR II, and FACSCaiibur™ (BD Biosciences, San Jose, Calif.) and other equivalent cell sorters produced by other commercial vendors such as Sony, Bio-Rad, and Beckman Coulter.

Alternatively, T-cells comprising peptides efficiently presented by the MHC may be enriched by MACS-based cell sorting.

“MACS” refers to a method of separating a population of cells into one or more sub-populations based on the presence, absence, or level of one or more MACS-selectable polypeptides expressed by the cells. MACS relies on magnetic susceptibility properties of tagged individual cells in order to sort the cells into sub-populations. For MACS, magnetic beads (such as those available from Miltenyi Biotec Bergisch Gladbach, Germany; 130-048-402) can be used as labels. MACS cell sorters suitable for carrying out a method described herein are well-known in the art and commercially available. Exemplary MACS cell sorters include autoMACS Pro Separator (Miltenyi Biotec).

The sorting results in a population of non-fluorescent cells and at least one population of fluorescent cells, depending on how many fluorescent labels were used. The presence of at least one cell population with fluorescent cells is indicative that at least one candidate peptide is efficiently presented by APCs. Thus, FACS enables sorting of the population of cells to produce a population of cells enriched in T-cells comprising peptides efficiently presented by the MHC.

The sequence of the TCRs and the corresponding cognate peptides can be obtained by single cell RNA/DNA sequencing of such a population of enriched T-cells.

Methods of DNA isolation and sequencing are known to those skilled in the art.

In general, the aim is to separate DNA present in the nucleus of the cell from other cellular components. The isolation of DNA usually begins with lysis or breakdown of cells. This process is essential for the destruction of protein structures and allows for release of nucleic acids from the nucleus. Lysis is carried out in a salt solution, containing detergents to denature proteins or proteases (enzymes digesting proteins), such as Proteinase K, or in some cases both. It results in the breakdown of cells and dissolving of membranes. Methods of DNA isolation include, but are not limited to, phenol:chloroform extraction, high salt precipitation, alkaline denaturation, ion exchange column chromatography, resin binding, and paramagnetic bead binding.

Methods of cDNA generation known to those skilled in the art. In general, the aim is to convert the isolated RNA present in the cells to DNA, co called copy-DNA, in order to use it as template for polymerase chain reaction (PCR). The isolation of RNA usually begins with lysis or breakdown of cells. This process is essential for the destruction of protein structures and allows for release of nucleic acids from it, Lysis is usually carried out in Phenol containing solution (e.g. TRIzol™). It results in the breakdown of cells and dissolving of membranes and allows the separation of RNA from other cellular components. The isolated RNA is then converted into cDNA by reverse transcriptase (e.g. Superscript™, Goscript™)

The sequence of the candidate peptides carried by the activated T cells (which bind to the MHC complexes presented on the antigen presenting cell surface) is then amplified by PCR and may be sequenced by any method known in the art.

The sequence of the candidate peptides may be determined by digital PCR. Digital polymerase chain reaction (digital PCR, DigitalPCR, dPCR, or dePCR) is a refinement of conventional polymerase chain reaction methods that can be used to directly quantify and clonally amplify nucleic acids including DNA, cDNA or RNA.

Sequencing may also be performed using microfluidics. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets.

Sequencing may also be performed using Second Generation Sequencing (or Next Generation or Next-Gen), Third Generation (or Next-Next-Gen), or Fourth Generation (or N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology. Morozova and Marra provide a review of some such technologies in Genomics, 92: 255 (2008).

Prior to, following or concurrently with sequencing, nucleic acids may amplified. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reverse transcribed to DNA prior to amplification (e.g., RT-PCR), whereas other amplification techniques directly amplify RNA (e.g., TMA and NASBA).

The peptide identified and enriched in the method as described herein may be used in in vivo interventions such as vaccination, induction of immunological tolerance, blocking of TCRs and MHC-mediated toxin delivery, for immunogenicity testing and other in vitro T-cell reactivity tests.

In one embodiment, the vaccine is a tumor specific antigen (TSA)-based cancer vaccine.

The term “vaccination” or equivalents are well-understood in the art. For example, the term vaccination can be understood to be a process that increases a subject's immune reaction to antigen and therefore the ability to resist or overcome a disease.

A “vaccine” is to be understood as meaning a composition for generating immunity for the prophylaxis and/or treatment of diseases (e.g. cancer). Accordingly, vaccines are medicaments which comprise antigens and are intended to be used in humans or animals for generating specific defense and protective substance by vaccination. The term “TSA-based cancer vaccine” is meant to refer to a vaccine containing a pooled sample of tumor-specific antigens, for example at least one, at least two, at least three, at least four, at least five, or more tumor-specific peptides.

Recurrent tumor-derived mutations may serve as public tumor-specific antigens enabling the development of TSA-based cancer vaccines applicable to broader patient cohorts. Accordingly, the method of the present invention can be used for identifying patients efficiently presenting these common/public TSAs to the immune system and potentially leading to efficient immune responses. However, many tumor-derived mutations appear to derive from patient-specific alterations. Thus, the method of the present invention can also be used for identifying patient-specific candidate peptides for personalized vaccines.

As used herein, “immunological tolerance” refers to a reduction in immunological reactivity of a host towards a specific antigen or antigens. The antigens comprise immune determinants that, in the absence of tolerance, cause an unwanted immune response. Immunological tolerance can be induced to prevent or ameliorate transplant rejection, autoimmunity, allergic reaction, or another undesirable immune response.

“Blocking of TCRs” refers to any agent which includes a peptide-MHC complex or p-MHC-specific antibody which blocks natural TCR-MHC interactions. “MHC-mediated toxin delivery” refers to methods covalently linking toxic agents (proteins or other) to peptide-MHC tetramers or other MHC multimers in order to deliver the said toxin into the cell to cause the death of the cell.

The term “immunogenicity testing” as used herein refers to measuring the potential immune responses to biotherapeutics. Biotherapeutics can elicit an immune response that may impact their safety and efficacy. Immunogenicity testing is employed to monitor and evaluate humoral (antibody) or cellular (T cells) responses during clinical and pre-clinical studies. Usually testing immunogenicity of a biotherapeutics involves measuring antibodies specifically generated against the biotherapeutics. With the method of the present invention it is possible to identify peptides that are efficiently presented by MHC molecules and is recognized by T cell so can potentially elicit an immune response. This can help to provide a fuller picture of the overall immunogenic profile of a compound.

The term “T-cell reactivity” as used herein refers to the capability of a substance to elicit T-cell activation. More specifically, “T-cell reactivity” means the capability of a peptide to induce proliferation or cytokine production of T cells.

The methods of the present invention may also be applied to high throughput screening. High throughput screening (HTS) technology is commonly used to define the rapid processing of cells on a large scale. In certain embodiments, a plurality of screens may be run in parallel with different candidate peptide libraries. high throughput screening systems are commercially available and typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization.

By the term “peptide” as used herein is meant at least two covalently attached amino acids. Generally, peptides attached to the N-terminus of the co-receptor can vary from 7 amino acids to 30 amino acids or more, particularly from 15 to 24 amino acids in length, particularly from 7 to 10 amino acids in length.

The term “antigen” as used herein refers to all, or parts, of a peptide or protein, capable of eliciting an immune response against itself or portions thereof. This immune response may involve either antibody production, or the activation of specific immunologically competent cells, or both.

The term “library” or equivalents as used herein means a plurality of molecules. In the case of peptides to be attached to the CD4, LAG-3 or CD8 co-receptor protein, the library provides a sufficiently structurally diverse population of peptides to effect a probabilistically sufficient range of cellular responses to provide one or more cells exhibiting a desired response. In a preferred embodiment, at least 7, preferably at least 50, more preferably at least 200 and most preferably at least 1000 peptides are simultaneously analyzed in the method of the invention. Libraries can be designed to maximize library size and diversity.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. Recombinant nucleic acid, is originally formed in vitro, in general, by the manipulation of nucleic acid, e.g., using polymerases and endonucleases, in a form not normally found in nature. In this manner, operably linkage of different sequences is achieved. Thus, an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a recombinant protein, such as the MHC-peptide complex of the invention, is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

Furthermore, in the claims the word “comprising” does not exclude other elements or steps, and the indefinite article “a”, “an”, and “the” include plural referents unless the context clearly dictates otherwise.

Unless otherwise defined, all technical and scientific terms used herein have the same meaning as commonly understood by one of ordinary skilled in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Specific embodiments of the present invention are additionally illustrated by the following examples. However, it should be understood that the invention is not limited to the specific details of these examples. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

The figure provided below, illustrates the structure of the chimeric receptors and provides a proof of concept in mice. The method according to the invention and as described herein identifies natural epitopes of T-cells in an unbiased and efficient manner. It holds promise for basic research and clinical applications allowing multidimensional, high-throughput personalised identification of T cell antigens in patients.

FIG. 1: Proof of concept for PEP4. a) Shown are: the structure of the chimeric PEP4 receptor and a schematic of its interaction with the MHC, leading to peptide-MHC complex recognition by the TCR. b) CD4-negative T-cell hybridomas, carrying a nur77 fluorescent reporter, were derived from Smarta2 T-cells (gp61-specific) or 2D2 T-cells(NFM-specific) and transduced with a construct encoding GFP and PEP4 carrying the gp61 peptide (PEP4gp61iresGFP). PEP4gp61 was efficiently expressed on the cell surface as measured by CD4-specific antibody staining shown in the dot plot. c,d) The Smarta2 hybridoma was transduced with gp61 linked to CD4 or CD3 with a GS-linker ranging from 12 to 28 amino acids and cultured with BMDCs from C57BL/6 (c,d) or BALB/c (d). e) The Smarta2 and 2D2 hybridomas were transduced with constructs encoding PEP4 receptors carrying gp61, OVA or NFM peptides and GFP and cultured with BMDCs from C57BL/6. c, d, e) Activation (nur77-reporter signal) was measured by FACS. Peptides were recognized in a specific and MHC-restricted manner and only those attached to CD4, but not to CD3, could be efficiently presented by the MHC. f) CD4+ Smarta2 or B6 T-cells were stimulated with anti-CD3/CD28 for 24 h, transduced with PEP4gp61iresGFP, taken of anti-CD3/CD28 and 48 h post-infection co-cultured with B6 BMDCs for several days. The graph shows fraction GFP-positive (expressing PEP4gp61) cells normalised to transduction efficiency. Day2 of co-culture corresponds to day4 post transduction. Smarta2 T cells, but not polyclonal B6 T cells, carrying PEP4gp61 were progressively enriched in culture, while it was not the case for cells transduced with the control peptide invNFM.

FIG. 2: Proof of concept for CD4-positive cells. CD4+ Sm2 hybridoma cells were infected with pMY-CD4gp61iresGFP or pMY-CD4OVAiresGFP. Two days later cells were cocultured for 9 h with B6 BMDCs (n=4 wells). After staining with anti-TCR, anti-CD4 and anti-CD69 antibodies, the activation of Sm2 hybridoma cells was measured through CD69 expression determined by FACS analysis. Dot plots in A) show TCR, CD4 and GFP expression in transduced cells, histogram in B) CD69 expression on TCR+CD4+GFP+ cells carrying PEP4-gp61 or PEP4-OVA or TCR+CD4+GFP− cell from the gp61 transduction, C) shows summary of CD69 expression (MFI). T test results, are indicated, **=0.0019. ****<0.0001.

### EXAMPLE 1

Materials and Methods

Cells

CD4⁺ T cells were isolated fro C57BL/6J, Smarta and 202 mice by FACS.

Flow Cytometry

The following antibodies were used: Fc block (anti-CD16/CD32; 2.4G2; home-made); CD4-APC (GK1.5), CD4-PE (GK1.5). Cells were analyzed on FACSCanto II or LSRFortessa (BD Bioscience) and data were analyzed in FlowJo software (Tree Star).

Hybridoma Generation

Sorted T-cells were activated with plastic-bound anti-CD3c and anti-CD28 antibodies in the presence of mouse IL-2 for 2-3 days. Equal numbers of activated T-cells and the TCRα⁻β⁻ BW5147 fusion partner were fused using PEG-1500, and plated at limiting dilution in the presence of 100 mM hypoxanthine, 400 nM aminopterin, and 16 mM thymidine (HAT).

Cloning of the PEP4 and PEP3 Constructs

DNA fragments encoding the gp61 or NFM peptides were inserted between the leader peptide and the GS-linker (ranging from 12 to 28 amino acids) connected to the rest of the full length CD4 or CD3 molecules and cloned into the pMYsiresGFP retroviral vector.

Retroviral Transduction of Reporter Cell Lines and Sorted Thymocytes

Retrovirus containing supernatants were produced in the ecotropic Phoenix packaging cell line and used to infect reporter cell lines and sorted cells activated with anti-CD3/CD28 for 24 h. For the transduction with nur77-reporter and PEP4 constructs CD4⁻ variants of the hybridomas were selected.

Stimulation of PEP4⁺ and PEP3⁺ Hybridomas with Bone Marrow-Derived Dendritic Cells

GFP+ cells were co-cultured with a >3-fold excess of bone marrow-derived dendritic cells for 8-12 h and reporter activation was measured no FACS.

The results are shown in the figures. That is, a proof of concept is shown based on PEP4. Specifically, a structure of the chimeric PEP4 receptor and a schematic of its interaction with the MHC, leading to peptide-MHC complex recognition by the TCR is shown in part a). As can be seen from part b) of the figure CD4-negative T-cell hybridomas, carrying a nur77 fluorescent reporter, were derived from Smarta2 T-cells (gp61-specific) or 202 T-cells(NFM-specific) and transduced with a construct encoding GFP and PEP4 carrying the gp61 peptide (PEP4gp61iresGFP). PEP4gp61 was efficiently expressed on the cell surface as measured by CD4-specific antibody staining shown in the dot plot. The Smarta2 hybridoma was transduced with gp61 linked to CD4 or CD3 with a GS-linker ranging from 12 to 28 amino acids and cultured with BMDCs from C57BL/6 (c,d) or BALB/c (d). The Smarta2 and 2D2 hybridomas were transduced with constructs encoding PEP4 receptors carrying gp61, OVA or NFM peptides and GFP and cultured with BMDCs from C57BL/6. c, d, e) Activation (nur77-reporter signal) was measured by FACS.

As is evident, peptides were recognized in a specific and MHC-restricted manner and only those attached to CD4, but not to CD3, could be efficiently presented by the MHC. Moreover, CD4+ Smarta2 or B6 T-cells were stimulated with anti-CD3/CD28 for 24 h, transduced with PEP4gp61 iresGFP, taken of anti-CD3/CD28 and 48 h post-infection co-cultured with B6 BMDCs for several days (part f). The graph shows the fraction of GFP-positive (expressing PEP4gp61) cells normalized to transduction efficiency. Day2 of co-culture corresponds to day4 post transduction. Smarta2 T-cells, but not polyclonal B6 T-cells, carrying PEP4gp61 were progressively enriched in culture, while it was not the case for cells transduced with the control peptide invNFM, Accordingly, it has surprisingly and unexpectedly been shown that a chimeric molecule comprising the CD4, MAG3 or CD8 co-receptor and a peptide attached to the N-terminus of the co-receptor can be employed to identify T-cell specific antigens, as claimed.

1. A chimeric molecule comprising the CD4, LAG3 or CD8 co-receptor protein and a peptide attached to the N-terminus of the co-receptor.

2. The chimeric molecule of claim 1, wherein the peptide or a part of the peptide can be presented by a major histocompatibility complex.

3. The chimeric molecule of claim 1, wherein the peptide:
(a) has a length of 6 to 200 amino acid residues;
(b) has a length of 7 to 30 amino acid residues;
(c) is a random peptide;
(d) is encoded by a given DNA or cDNA molecule;
(e) is derived from a tumor cell or from a cell that has been infected with a pathogen;
(f) comprises an epitope of a tumor antigen;
(g) comprises an amino acid sequence with at least 50%, 60%, 70%, 80%, 90% sequence identity with an epitope of a tumor antigen;
(h) comprises an MHC class I epitope when the co-receptor protein is CD8; and/or
(i) comprises an MHC class II epitope when the co-receptor protein is CD4 or LAG3.

4-6. (canceled)

7. The chimeric molecule of claim 3, wherein the DNA or cDNA molecule encoding the peptide is obtained by fragmentation of a larger DNA or cDNA molecule.

8-9. (canceled)

10. The chimeric molecule of claim 3, wherein the tumor antigen is a neoantigen.

11-13. (canceled)

14. The chimeric molecule of claim 1, wherein the CD4 co-receptor protein is a human CD4 co-receptor protein, the LAG3 co-receptor protein is a human LAG3 co-receptor protein and the CD8 co-receptor protein is a human CD8 co-receptor protein.

15. The chimeric molecule of claim 1, wherein the peptide is attached to the N-terminus of the co-receptor via a linker.

16. The chimeric molecule of claim 15, wherein the linker:
(a) has a length between 5 and 30 amino acids; and/or
(b) comprises the amino acid sequence (GGGGS)ₓ, wherein G is glycine, S is serine and x is the number of repetitions, wherein x can be any number between 1 and 5.

17. The chimeric molecule of claim 15, wherein at least 40%, at least 50%, at least 60%, at least 70%, at least 80%, at least 90% of the amino acid residues in the linker are glycine or serine residues.

18. (canceled)

19. A polynucleotide encoding the chimeric molecule of claim 1.

20. A library of polynucleotides comprising a plurality of polynucleotides of claim 1.

21. The library of polynucleotides of claim 20, wherein at least two polynucleotides of the library encode an identical co-receptor protein attached to a different peptide.

22. A cell comprising the polynucleotide of claim 19.

23. A method for simultaneously identifying antigen-specific T cell receptors and the peptides specifically recognized by said T cell receptors (TCRs), the method comprising the steps of:
(a) providing polyclonal T cells of interest expressing the library of polynucleotides of claim 20;
(b) contacting the T cells of step (a) with antigen presenting cells (APC) comprising a major histocompatibility complex (WIC);
(c) isolating at least one T cell that is activated upon contacting with the APCs in step (b);
(d) sequencing the DNA of the isolated T cells of step (c) to obtain information about the TCR sequences and the peptide sequences attached to the CD4, LAG-3 or CD8 co-receptors present in these T cells; and
(e) identifying cognate T cell receptor-peptide pairs based on the sequencing data obtained in step (d).

24. A method for identifying at least one antigen-specific T cell receptor, the method comprising the steps of:
(a) providing polyclonal T-cells of interest expressing a polynucleotide of claim 19;
(b) contacting the T-cells of step (a) with antigen presenting cells (APC) comprising a major histocompatibility complex (MHC);
(c) isolating at least one T-cell that is activated upon contacting with the APCs in step (b);
(d) sequencing of the TCR loci of the at least one T cell isolated in step (c); and
(e) identifying at least one T cell receptor encoded by the TCR loci of the at least one T cell to be antigen-specific.

25. A method for identifying at least one T cell-specific antigen, the method comprising the steps of:
(a) providing monoclonal T-cells of interest expressing a polynucleotide of claim 19;
(b) contacting the T cells of step (a) with antigen presenting cells (APC) comprising a major histocompatibility complex (MHC);
(c) isolating at least one T cell that is activated upon contacting with the APCs in step (b);
(d) sequencing the part of the polynucleotide encoding the peptide attached to the N-terminus of a CD4, LAG-3 or CD8 co-receptor protein of the at least one T cell isolated in step (c); and
(e) identifying at least one peptide encoded by the polynucleotide comprised in the at least one T cell to be a T cell-specific antigen.

26. The method of claim 23, wherein the APC is an autologous or a heterologous APC.

27. The method of claim 23, wherein the APC is a genetically modified autologous or heterologous cell or cell line, expressing a mutated MHC molecule.

28. The method of claim 27, wherein the mutated MHC molecule is:
(a) a MHC class II molecule comprising the extracellular MHC class II alpha chain and a native or heterologous transmembrane domain, as well as the extracellular MHC class II beta chain and a native or heterologous transmembrane domain; or
(b) a MHC class I molecule comprising the extracellular MHC class I alpha chain and a native or heterologous transmembrane domain, as well as beta-2 microglobulin.

29. (canceled)

30. The method of claim 23, wherein the co-receptor protein encoded by the polynucleotide is:
(a) CD8 if the MHC molecule comprised in the APC is a MHC class I molecule; or
(b) CD4 or LAG-3 if the MHC molecule expressed by the APC is a MHC class II molecule.

31. (canceled)

32. A method for treating a subject suffering from cancer, the method comprising the steps of:
(a) Identifying at least one antigen-specific T cell receptor and/or at least one T cell-specific antigen with the method of claim 23;
(b) administering to the subject suffering from cancer the at least one T cell receptor and/or T cell-specific antigen identified in step (a).

33. The method of claim 32, wherein the antigen-specific T cell receptor is administered to the subject by virus-mediated gene delivery.

34. The method of claim 32, wherein the T cell-specific antigen is administered to the subject in form of a peptide or in form of a polynucleotide encoding a peptide.

35. The method of claim 34, wherein the peptide or the polynucleotide encoding the peptide is attached to a compound that improves delivery of the peptide or polynucleotide encoding the peptide to an APC.


SEQUENCE LISTING (2 sequences)

SEQ ID NO: 1 - protein, 12 aa, Artificial Sequence
  12 amino acid linker
GSGGGGSGGGGS

SEQ ID NO: 2 - protein, 15 aa, Artificial Sequence
  15 amino acid linker
GSGGSGGGGSGGGGS